(19) United States
(12) Patent Application Publication
(10) Pub. No.: US 2007/0015279 A1
(43) Pub. Date: Jan. 18, 2007

(54) Methods of Selecting Pancreatic Endocrine Cells Using Protein Synthesis Inhibitors

(75) Inventors: Wen-Ghih Tsang, Sherman Oaks, CA (US); Hong-Jung Chen, Arcadia, CA (US); Yiping Tu, Los Angeles, CA (US); Yanping Wang, Los Angeles, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: AmCyte Inc.

(21) Appl. No.: 11/419,093

(22) Filed: May 18, 2006

Related U.S. Application Data
(60) Provisional application No. 60/682,473, filed on May 18, 2005.

(51) Int. Cl. C12N 5/08; C12M 3/00

(52) U.S. Cl. 435/366; 435/289.1

(57) ABSTRACT

The present invention relates to methods of selecting pancreatic endocrine cells from total pancreatic cells by incubation with protein synthesis inhibitors.

### CROSS-REFERENCES TO RELATED APPLICATIONS

This application claims the benefit of U.S. Provisional Patent Application No. 60/682,473, filed May 18, 2005, the teachings of which are herein incorporated by reference.

### BACKGROUND OF THE INVENTION

Human islet transplantation is a promising treatment for diabetic patients who need insulin supplementation. However, two challenges must be overcome before this treatment can replace insulin injection: the shortage of donor organs and the toxicity of chronic immunosuppression. Methods for immune isolation of islet cells, such as encapsulation, have helped to decrease the need for immunosuppressive therapy, while the shortage of donor organs remains an intractable problem.

Matured or terminally differentiated endocrine cells, such as adult brain cells, are considered to be post-mitotic cells and are believed to have very low or no mitotic activity. Anatomically, pancreatic endocrine cells are organized into discrete structural and functional units called Islet of Langerhans. The main function of pancreatic endocrine cells is secretion of hormones to maintain homeostasis of blood glucose levels. Each Islet of Langerhan is surrounded by exocrine acinar cells. The major function of exocrine cells is secretion of enzymes such as amylase, lipases and proteases into collecting ducts that are subsequently transport to stomach for food digestion. In a human adult, the Islets of Langerhan constitute about 5% of the total mass of pancreas while exocrine and ductal tissues make up the majority of total pancreas mass.

Progenitors of pancreatic endocrine cells are few in number and quiescent in intact pancreas. Methods to efficiently isolate such progenitor cells from donor pancreas have been unsuccessful to date. The present invention solves this and other problems.

### BRIEF SUMMARY OF THE INVENTION

In one aspect, the present invention provides a method of isolating a culture of pancreatic endocrine lineage cells, by (a) isolating a culture of pancreatic cells from a pancreas, which includes a population of pancreatic endocrine lineage cells and a population of pancreatic exocrine cells; (b) incubating the culture of isolated pancreatic cells with an inhibitor of protein synthesis in an amount that is lethal to at least 30% of the population of pancreatic exocrine cells; and (c) isolating the population of pancreatic endocrine lineage cells that survive incubation with the inhibitor of protein synthesis to obtain the culture of pancreatic endocrine lineage cells.

In one embodiment the inhibitor of protein synthesis is an antibiotic. In a further embodiment, the inhibitor of protein synthesis is one of the following: hygromycin B, puromycin, and G418.

In one embodiment, at least 40% of the isolated pancreatic endocrine lineage cells express CD56 as a cell surface marker. In another embodiment, at least 80% of the isolated pancreatic endocrine lineage cells express CD56 as a cell surface marker. In a further embodiment, at least 90% of the isolated pancreatic endocrine lineage cells express CD56 as a cell surface marker.

In one embodiment, the culture of isolated pancreatic cells is incubated with an amount of the inhibitor of protein synthesis that kills at least 50% of the population of pancreatic exocrine cells. In another embodiment, the culture of isolated pancreatic cells is incubated with an amount of the inhibitor of protein synthesis that kills at least 90% of the population of pancreatic exocrine cells. In a further embodiment, the culture of isolated pancreatic cells is incubated with an amount of the inhibitor of protein synthesis that kills at least 95% of the population of pancreatic exocrine cells.

In one embodiment, the culture of pancreatic endocrine cells comprises a stromal cell.

In one embodiment, the culture of isolated pancreatic cells is isolated from a human pancreas.

In one embodiment, the culture of pancreatic endocrine lineage cells is expanded, i.e., the cells are allowed to proliferate. In a further embodiment, the expanded culture of pancreatic endocrine lineage cells is differentiated. In a still further embodiment, the differentiated culture of pancreatic endocrine lineage cells is encapsulated.

In one aspect the invention provides a method of providing pancreatic endocrine function to a mammal in need of such function, by (a) isolating a culture of pancreatic cells from a pancreas, which includes a population of pancreatic endocrine lineage cells and a population of pancreatic exocrine cells; (b) incubating the culture of isolated pancreatic cells with an inhibitor of protein synthesis in an amount that is lethal to at least 30% of the population of pancreatic exocrine cells; (c) isolating the population of pancreatic endocrine lineage cells that survive incubation with the inhibitor of protein synthesis to obtain the culture of pancreatic endocrine lineage cells, and (d) implanting into the mammal the culture of pancreatic endocrine lineage cells in an amount sufficient to produce a measurable amount of insulin in the mammal.

In some embodiments the following additional steps are performed: (i) expanding the culture of pancreatic endocrine lineage cells; (ii) differentiating the expanded culture of pancreatic endocrine lineage cells; and (iii) encapsulating the differentiated culture of pancreatic endocrine lineage cells, before implanting the differentiated culture of pancreatic endocrine lineage cells into a mammal.

In one embodiment, the culture of pancreatic endocrine lineage cells is implanted into a human. In another embodiment implantation of the culture of pancreatic endocrine lineage cells is used treat diabetes in a mammal in need of such treatment.

In one aspect, the present invention provides a culture vessel with (a) a tissue culture medium, (b) a predetermined amount of an exogenously added protein synthesis inhibitor, and (c) a culture of untransfected pancreatic endocrine lineage cells. Untransfected pancreatic endocrine lineage cells are cells that do not contain an exogenous nucleic acid molecule, e.g., cells that have not been transfected with a recombinant DNA molecule or infected with a recombinant virus. In particular, cells that are transfected with an exogenous nucleic acid that maintained by the presence of a protein synthesis inhibitor in the medium.

In one embodiment, the inhibitor of protein synthesis is selected from the group consisting of hygromycin B, puromycin and G418.

In another embodiment, the pancreatic endocrine cells are human cells.

### BRIEF DESCRIPTION OF THE DRAWINGS

FIG. 1 provides in vivo function of HD451B P4 cells.

FIG. 2 provides SGS results from retrieved MICMACs originally cultured in DM (panel A) or M4 (panel B). Results are expressed as means and standard deviation from triplicate samples (9 MICMACs in each) with three measurements. L1, L2, L3: glucose 60 mg/dL. H: glucose 450 mg/dL+theophyllin 10 mM.

### DEFINITIONS

As used herein, a “pancreatic endocrine lineage cell” refers to a cell isolated from the islets of langerhans of a pancreas, or the progeny of such a cell. Pancreatic endocrine cells express and secrete hormones, e.g. insulin and glucagons, into the bloodstream. Typically, pancreatic endocrine lineage cells are isolated from a donor. A “culture of pancreatic endocrine lineage cells” or a “cell culture of pancreatic endocrine cells” refers to pancreatic endocrine lineage cells or their progeny that are grown outside the body of the original donor. Cultures of pancreatic endocrine lineage cells can include non-endocrine cells that support growth of endocrine cells, e.g., stromal cells.

Pancreatic endocrine lineage cells include cells at all stages of development. For example, pancreatic endocrine lineage cells include progenitor cells that are capable of division and that can differentiate into cells that express high levels of insulin. Pancreatic endocrine lineage cells also include proliferating pancreatic endocrine cells that are actively dividing. Pancreatic endocrine lineage cells also include mature pancreatic endocrine cells that can form insulin producing aggregates or are part of such aggregates and that secrete high levels of hormones, e.g., insulin and glucagon.

As used herein, “pancreatic cells from a pancreas” or “a culture of pancreatic cells from a pancreas” refers to a total cell population isolated from a donor pancreas and includes e.g., both pancreatic endocrine cells and pancreatic exocrine cells.

As used herein, “pancreatic exocrine cells” refers to pancreatic cells that secrete pancreatic enzymes for digestion into the gastrointestinal tract. Pancreatic enzymes include e.g., trypsin, chymotrypsin, and carboxypeptidase. Measurement of levels of pancreatic enzyme nucleic acids and proteins can thus be used to determine the presence or absence of pancreatic exocrine cells in a cell population. Pancreatic exocrine cells includes cells at all stages of development, e.g., progenitor cells, dividing cells, and mature enzyme secreting cells.

As used herein, an “inhibitor of protein synthesis” or a “protein synthesis inhibitor” is a compound that interferes with or selectively inhibits cellular synthesis of proteins, e.g., translation of proteins from mRNA. Protein synthesis can be inhibited by protein synthesis inhibitors by a variety of mechanisms, including e.g., perturbation of initiation, binding to ribosomal components to block binding to tRNAs, inhibition of peptidyl transferase activity, inhibition of translocation, and premature termination. Such inhibitors are distinguished from other toxins such as cyanide or lethal agents such as heat or cold, by their selective activity against the above mechanisms. Assays defining protein synthesis inhibitors are provided below. In some embodiments the protein synthesis inhibitors are also antibiotics. Many commercially available antibiotics are protein synthesis inhibitors and can be used in the methods of the invention to select pancreatic endocrine cells. Examples of antibiotics that are protein synthesis inhibitors include e.g., hygromycin, G418, amikacin, gentamicin, kanamycin, neomycin, netilmicin, paromomycin, streptomycin, and puromycin.

A “lethal amount” of a protein synthesis inhibitor or “an amount that is lethal” is an amount of a protein synthesis inhibitor that kills a cell, whether by apoptotic or necrotic death. In some embodiments, a lethal amount of a protein synthesis inhibitor will kill at least 20, 25, 30, 40, 50, 60, 60, 70, 80, 85, 90, 95, 96, 97, 98 or 99% of pancreatic exocrine cells in a population of pancreatic cells from a pancreas. In other embodiments, a lethal amount of a protein synthesis inhibitor will kill at least 20, 25, 30, 40, 50, 60, 60, 70, 80, 85, 90, 95, 96, 97, 98 or 99% of all pancreatic cells in a population of pancreatic cells from a pancreas. In preferred embodiments, some or most of the surviving cells are pancreatic endocrine lineage cells.

As used herein, “CD56 protein” refers to a cell surface glycoprotein thought to play a role in embryogenesis, development, and contact mediated interactions between cells. Because of differential transcript splicing, the majority of CD56 protein are found in three major sizes: 180 kDa, 140 kDa, and 120 kDa. Exemplary CD56 proteins include human CD56 proteins, for example the 120 kDal form, Accession Number P13592; the 140 kDal form, Accession Number P13591, and the 180 kDal form, see e.g., Hemperly, J. et al, J. Mole Neurosci. 2:71-78 (1990).

The term “CD56 binding reagent” is used herein to refer to a compound that specifically binds to a CD56 protein or to molecules covalently linked to a CD56 protein, such as oligosaccharides. In a preferred embodiment, the CD56 binding reagent is an antibody that specifically binds to the CD56 protein. The term “CD56 binding reagent” also encompasses compounds that are specifically bound by the CD56 protein, for example heparin and heparin sulfate. The term encompasses ligands and lectins as defined herein. CD56 binding reagents are used to identify or select cells that express CD56 protein as a cell surface marker.

Cells that “exhibit CD56 as a cell surface marker” are cells that exhibit a sufficient quantity of CD56 on the cell surface to allow the cells to be selected or picked out from a population of cells using conventional CD56 specific binding reagents and methods described herein, such as FACS, immunocytochemistry, immunoadsorbtion, and panning. In a preferred embodiment a CD56 antibody is used to select cells that “exhibit CD56 as a cell surface marker.”

“Insulin:actin mRNA ratios” are measured by hybridization or amplification assays using labeled nucleic acid probes. Labeled product is determined using gel scanner or by real time PCR using different labeled probes for insulin and actin. With these methods, insulin:actin mRNA ratios are an average across a population of cells. Insulin:actin mRNA rations can also be measured on an individual cell basis using in situ hybridization. In some embodiments, insulin:actin ratios are determined to identify pancreatic endocrine lineage cells. For such determinations, preferred insulin:actin ratios are e.g., between 0.001 and 10,000; between 0.01 and 1,000; or between 0.1 and 100. The ratios will vary with the developmental stage of the pancreatic endocrine lineage cell. For example, pancreatic endocrine lineage progenitor cells will exhibit lower insulin:actin ratios. In contrast, mature, insulin producing pancreatic endocrine lineage cells will have insulin:actin ratios in the upper ranges.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al, Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al, Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to CD56 proteins, polymorphic variants, alleles, orthologs, and conservatively modified variants, or splice variants, or portions thereof, can be selected to obtain only those polyclonal antibodies that are specifically immunoreactive with CD56 proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Specific binding can also be used to describe the interaction of other molecules that specifically bind to CD56 protein, e.g. CD56 ligands and lectins that recognize CD56.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

As used herein, “insulin producing cells” refers to cells that secrete detectable amounts of insulin. “Insulin producing cells” can be individual cells or collections of cells. One example of a collection of “insulin producing cells” is “insulin producing cell aggregates” e.g., an organized collection of cells with a surrounding mantle of CK-19 positive cells and an inner cell mass. “Aggregate” in the context of cells refers to a three dimensional structure. “CK-19” is a 40 kD acidic keratin, cytokeratin 19. “Mantle” refers to an envelope of cells surrounding in three dimensions the inner cell mass.

The term “contacting” is used herein interchangeably with the following: combined with, added to, mixed with, passed over, incubated with, flowed over, etc.

“Differentiate” or “differentiation” refers to a process where cells progress from an undifferentiated state to a differentiated state or from an immature state to a mature state. For example, undifferentiated pancreatic cells are able to proliferate and express characteristics markers, like PDX-1. Mature or differentiated pancreatic cells do not proliferate and secrete high levels of pancreatic endocrine hormones. E.g., mature β-cells secrete insulin at high levels. Changes in cell interaction and maturation occur as cells lose markers of undifferentiated cells or gain markers of differentiated cells. Loss or gain of a single marker can indicate that a cell has “matured or differentiated.”

The term “differentiation factors” refers to a compound added to pancreatic cells to enhance their differentiation to mature insulin producing β cells. Exemplary differentiation factors include hepatocyte growth factor, keratinocyte growth factor, exendin-4, basic fibroblast growth factor, insulin-like growth factor-I, nerve growth factor, epidermal growth factor and platelet-derived growth factor.

The term “providing pancreatic function to a mammal in need of such function” refers to a method of producing pancreatic hormones within the body of a mammal unable to produce such hormones on its own. In a preferred embodiment, insulin is produced in the body of a diabetic mammal. The pancreatic function is provided by implanting or transplanting aggregates of insulin producing pancreatic cells, produced by the methods of this disclosure into the mammal. The number of aggregates implanted is an amount sufficient to produce a measurable amount of insulin in the mammal. The insulin can be measured by Western blotting or by other detection methods known to those of skill in the art, including assays for insulin function, such as maintenance of blood glucose levels. Insulin can also be measured by detecting C-peptide in the blood. In another preferred embodiment, the provision of pancreatic function is sufficient to decrease or eliminate the dependence of the mammal on insulin produced outside the body.

“Encapsulation” refers to a process where cells are surrounded by a biocompatible acellular material, such as sodium alginate and polylysine. Preferably small molecules, like sugars and low molecular weight proteins, can be taken up from or secreted into an environment surrounding the encapsulated cells. At the same time access to the encapsulated cells by larger molecules and immune cells is limited.

“Implanting” is the grafting or placement of the cells into a recipient. It includes encapsulated cells and non-encapsulated. The cells can be placed subcutaneously, intramuscularly, intraportally or interperitoneally by methods known in the art.

A “population” of cells refers to a plurality of cells obtained by a particular isolation or culture procedure. While the selection processes of the present invention yield populations with relatively uniform properties, a population of cells may be heterogeneous when assayed for marker expression or other phenotype. Properties of a cell population are generally defined by a percentage of individual cells having the particular property (e.g., the percentage of cells staining positive for a particular marker) or the bulk average value of the property when measured over the entire population (e.g., the amount of mRNA in a lysate made from a cell population).

“Passage” of cells usually refers to a transition of a seeded culture container from a partially confluent state to a confluent state, at which point they are removed from the culture container and reseeded in a culture container at a lower density. However, cells may be passaged prior to reaching confluence. Passage typically results in expansion of the cell population as they grow to reach confluence. The expansion of the cell population depends on the initial seeding density but is typically a 1 to 10, 1 to 5, 1 to 3, or 1 to 2 fold expansion. Thus, passaging generally requires that the cells be capable of a plurality of cell divisions in culture.

### DETAILED DESCRIPTION OF THE INVENTION

I. Introduction

This disclosure is based at least in part on the surprising finding that pancreatic endocrine lineage cells can be selected from a population of total pancreatic cells using protein synthesis inhibitors. In some embodiments, pancreatic endocrine lineage cells proliferate during or after the protein synthesis inhibitor selection. Thus, the methods of the invention include a cell culture process which selects and proliferates pancreatic endocrine cells.

Some pancreatic endocrine lineage cells exist in the pancreas in a quiescent state and enter a process of differentiation and/or proliferation after receiving appropriate stimuli. These pancreatic endocrine lineage cells can survive with minimal protein synthesis capacity and are less sensitive to protein synthesis inhibitors than other pancreatic cells that need active protein synthesis for growth and cell division. After in vitro selection using protein synthesis inhibitors, pancreatic endocrine lineage cells can be maintained in culture, or frozen for later use, or induced to differentiate and/or proliferate, thereby becoming a culture of insulin producing pancreatic endocrine lineage cells. In some embodiments, the pancreatic endocrine lineage cells express the CD56 marker on their cell surface.

A wide variety of protein synthesis inhibitors can be used in the methods of the claimed invention. For example, antibiotics that inhibit protein synthesis can be used in the claimed methods. In one embodiment, hygromycin B, a selective agent in molecular genetic studies of a wide variety of prokaryotic and eukaryotic species, is used to select CD56 positive pancreatic progenitor cells. Hygromycin B is produced by Streptomyces hygroscopicus and belongs to the class of aminoglycoside antibiotics. It kills bacteria, fungi, and higher eukaryotic cells by inhibiting protein synthesis. It has been reported to inhibit translocation by sequestering tRNA in the ribosomal A site and to cause mistranslation. (Cabanas et al., Eur. J. Biochem., 87:21-27 (1978); Gonzalez et al., Biochim Biophys Acta, 521(2):459-69 (1978); and Singh et al., Nature, 277(5692):146-8 (1979)). The sensitivity of cells to hygromycin B toxicity is both species and cell-type dependent.

In another embodiment, puromycin is used in the methods of the claimed invention. Puromycin is an aminonucleoside antibiotic produced by Streptomyces alboniger. See, e.g., Rao et al., J. Biol. Chem. 244:112-8 (1969). The nucleoside part of puromycin resembles the aminoacyl-adenylyl terminus of aminoacyl-tRNA and competes for binding to the large ribosomal subunit. The incorporation of puromycin into a growing polypeptide causes termination. The antibiotic inhibits the growth of Gram positive bacteria and various animal and insect cells. Fungi and Gram negative are resistant due to the low permeability of the antibiotic. Puromycin also inhibits protein import into mitochondria by interfering with an ATP-dependent step in the import process and that the ATP-dependent component in the reaction is located inside the inner mitochondrial membrane. See, e.g., Price Verner Biochimica et Biophysica Acta 1150:89-97 (1993).

G418 or geneticin sulfate is an aminoglycoside antibiotic similar in structure to gentamicin, neomycin and kanamycin. It is used as a selective antibiotic in molecular biology and molecular genetics. G-418 is an analog of neomycin sulfate that interferes with the function of 80S ribosomes and protein synthesis in eukaryotic and prokaryotic cells.

Hygromycin B, puromycin, and G418 or geneticin sulfate were used to select pancreatic endocrine cells from human adult primary pancreatic cells. These selected cells were expanded and expressed insulin, NeuroD1, and other markers of pancreatic endocrine cells.

II. Isolation of Pancreatic Endocrine Cells

A variety of sources and methods can be used to isolate pancreatic endocrine cells.

A. Isolation of Pancreas from a Donor

Pancreatic cells isolated for subsequent culturing are obtained from one or more donated pancreases. The methods described herein are not dependent on the age of the donated pancreas. Accordingly, pancreatic material isolated from donors ranging in age from embryos to adults can be used.

In another embodiment, pancreatic cells are isolated from a cultured source. For example, cells prepared according to the microencapsulation method of U.S. Pat. No. 5,762,959 to Soon-Shiong, et al., entitled “Microencapsulation of cells,” can be harvested as a source of donor cells.

1. Isolation of Pancreatic Cells from Pancreas

Once a pancreas is harvested from a donor, it is typically processed to yield individual cells or small groups of cells for culturing using a variety of methods. One such method calls for the harvested pancreatic tissue to be cleaned and prepared for enzymatic digestion. Enzymatic processing is used to digest the connective tissue so that the parenchyma of the harvested tissue is dissociated into smaller units of pancreatic cellular material. The harvested pancreatic tissue is treated with one or more enzymes to separate pancreatic cellular material, substructures, and individual pancreatic cells from the overall structure of the harvested organ. Collagenase, DNAse, Liberase preparations (see U.S. Pat. Nos. 5,830,741 and 5,753,485) and other enzymes are contemplated for use with the methods disclosed herein.

Isolated source material can be further processed to enrich for one or more desired cell populations. However, unfractionated pancreatic tissue, once dissociated for culture, can also be used directly in the culture methods of the invention without further separation, and will yield the intermediate cell population. In one embodiment the isolated pancreatic cellular material is purified by centrifugation through a density gradient (e.g., Nycodenz, Ficoll, or Percoll). For example the gradient method described in U.S. Pat. No. 5,739,033, can be used as a means for enriching the processed pancreatic material in islets. The mixture of cells harvested from the donor source will typically be heterogeneous and thus contain α-cells, β-cells, δ-cells, ductal cells, acinar cells, facultative progenitor cells, and other pancreatic cell types.

A typical purification procedure results in the separation of the isolated cellular material into a number of layers or interfaces. Typically, two interfaces are formed. The upper interface is islet-enriched and typically contains 10 to 100% islet cells in suspension. The second interface is typically a mixed population of cells containing islets, acinar, and ductal cells. The bottom layer is the pellet, which is formed at the bottom of the gradient. This layer typically contains primarily (>80%) acinar cells, some entrapped islets, and some ductal cells. Ductal tree components can be collected separately for further manipulation.

The cellular constituency of the fractions selected for further manipulation will vary depending on which fraction of the gradient is selected and the final results of each isolation. When islet cells are the desired cell type, a suitably enriched population of islet cells within an isolated fraction will contain at least 10% to 100% islet cells. Other pancreatic cell types and concentrations can also be harvested following enrichment. For example, the culture methods described herein can be used with cells isolated from the second interface, from the pellet, or from other fractions, depending on the purification gradient used.

In one embodiment, intermediate pancreatic cell cultures are generated from the islet-enriched (upper) fraction. Additionally, however, the more heterogeneous second interface and the bottom layer fractions that typically contain mixed cell populations of islets, acinar, and ductal cells or ductal tree components, acinar cells, and some entrapped islet cells, respectively, can also be used in culture. While both layers contain cells capable of giving rise to the pancreatic endocrine cell population described herein, each layer may have particular advantages for use with the disclosed methods.

B. Isolation of Pancreatic Progenitor Cells Using Protein Synthesis Inhibitors

1. Defining protein synthesis inhibitors

The mechanism of protein synthesis or translation is well known in the art and is highly conserved across microbial, plant, and animal kingdoms. Protein synthesis is carried out on ribosomes. Briefly, protein synthesis includes three phases: initiation, elongation, and termination. Protein synthesis is well described in many textbooks, including e.g., Berg, Tymoczko, and Stryer, Biochemistry (5th ed. 2002); and Alberts et al., Molecular Biology of the Cell (4th ed., 2002).

Protein synthesis inhibitors of the instant invention are distinguished from other toxins or lethal agents by their selective activity to inhibit initiation and/or elongation of protein synthesis as further exemplified by the following in vitro assays. Such an assay will identify inhibitors of eukaryotic and prokaryotic protein synthesis that inhibit gene-specific expression at the level of mRNA-ribosome interaction by, for example, perturbing initiation, binding to ribosomal components to block binding to tRNAs, inhibiting peptidase transferase activity, inhibiting translocation, and/or causing premature termination.

One assay (see Novac et al. (2004) Nucleic Acid Research 32(3):902-915) is based on in vitro translation of a specific mRNA reporter that allows for the identification of inhibitors of cap-dependent translation; inhibitors that bind to a particular mRNA motif in the 5′-untranslated region (5′UTR) of the first cistron where they inhibit ribosome scanning; inhibitors of elongation or termination; and inhibitors of internal ribosomal entry segment (IRES)-mediated initiation. Cap-dependent translation/initiation is the major translation initiation pathway in eukaryotes, wherein the small (40S) ribosomal subunit requires several eukaryotic initiation factors (eIFs), first to bind initiator tRNA to form a 43S complex and then to bind mRNA to form a 48S complex. In comparison, a second, cap-independent mechanism of ribosome binding is used by mRNAs whose 5′UTR contains an IRES.

This assay quantitatively assesses the products of the firefly and renilla cistrons after in vitro translation of a test transcript. Specifically, any inhibitor that prevents 5′-mediated (cap-dependent) initiation or that binds to a particular RNA motif within the 5′-UTR and inhibits the 40S ribosome scanning will decreases expression of firefly luciferase, but will not affect renilla luciferase activity. Any inhibitor that prevents IRES-mediated initiation will decrease expression of renilla luciferase, but will not affect firefly luciferase activity. A general inhibitor of translation (i.e., initiation, elongation or termination) would reduce both firefly and renilla luciferase activity.

In a second in vitro assay (see Zemfira et al. (2003) Nucleic Acid Research 31(20):5949-5956), premature termination of translation can be tested by monitoring elongation release factors. More specifically, the assay utilizes translational frameshifting to assess translation termination. Generally, termination of protein synthesis occurs when the ribosome elongation machinery encounters an in-frame termination codon on the mRNA. In eukaryotes, there are two release factors that recognize the stop codons such as eukaryotic release factor 1 (eRF1), which recognizes all three stop codons UAG, UGA or UAA, and eukaryotic release factor 3 (eRF3). Translation termination normally occurs after completion of the full-length polypeptide.

In some genes, a termination signal can be alternatively decoded which causes translational frameshifting, readthrough or selenocysteine incorporation. This is referred to as “recoding”. This second in vitro assay monitors the activity of eukaryotic release factors in translation termination by measuring the relative frequency of competing frameshift events at the antizyme termination signal. Antizymes regulate the cellular level of polyamines (e.g., putrescine, spermidine and spermine) in a wide variety of eukaryotes. There are three members of the mammalian antizyme family, antizyme-1, 2 and 3. The initiating open reading frame (ORF1) of antizyme-1 mRNA has a UGA at codon 68 where the majority of translating ribosomes terminate translation, while the rest of the ribosomes undergo a +1 frameshift event to decode UCC UGA U sequence (where the terminator of ORF1 is underlined) to serine-aspartate and continue elongation along the second open reading frame (ORF2). Frameshifting is induced by polyamines. In the presence of an optimal concentration of polyamines, the frameshift efficiency is greater than 20 percent.

Thus, ribosomes decoding along the antizyme frameshift site will either terminate translation at the UGA codon, or undergo a +1 frameshift event and continue elongation. Therefore, the reduction in frameshift events corresponds to an increase in translation termination activity, and vice versa. The relatively high occurrence of antizyme frameshifting in rabbit reticulocyte lysates provides a convenient, quantitative assay to monitor release factor activity. In this assay system, termination at all three stop codons can be quantified by simply changing the codon in the termination signal of the tester mRNA. Since certain substances can mimic release factors (e.g., puromycin mimics release factors and causes premature termination). They can be tested in this system to assess their ability to cause premature termination. In fact, any substance could be tested for its ability to cause premature termination by measuring the number of frameshift events. A reduction in frameshift events corresponds to an increase in termination activity while an increase in frameshift events corresponds to a decrease in termination activity.

In a third in vitro assay (see Shelley et al. (2002) RNA 8:890-903) one can quantify the potency of puromycin or similar inhibitors to inhibit translation. Puromycin and its derivatives can inhibit translation in multiple ways. First, small puromycin derivatives can enter the peptidyl transferase center and become attached to the nascent protein chain in a factor-independent fashion. Derivatives that resemble tRNA can inhibit translation by sequestering soluble factors, like eukaryotic release factors. Puromycin attachment occurs predominantly at discrete locations within the template and near the end of the open reading frame. Second, puromycin can also function in a peptide-bond-independent mode. This inhibition is likely due to a combination of puromycin binding that blocks initiation, failure of ribosomes to recycle properly, or some other mode. In this third assay, a translation reaction is performed for 1 h using rabbit globin mRNA at about 60 nM (i.e., total template concentration, wherein the template is a mixture of α- and β-globin mRNAs) in rabbit reticulocyte lysate. Each reaction differs only in the amount of inhibitor that is added at the beginning of the reaction. The reactions are assayed by tricine-SDS PAGE, TCA precipitation, or other methods to determine the amount of globin translation product. This analysis results in a sigmoidal curve, and the midpoint (i.e., the concentration of drug required to give a 50% decrease in globin synthesis relative to a non inhibitor control) is termed the IC50. Globin synthesis is expected to decrease as the amount of puromycin is increased. More specifically, as the puromycin concentration is increased, a gradual decrease in the amount of the globin band is expected, with most of the remaining protein appearing as full-length globin. Since puromycin does not chase the protein into lower molecular weight species, two possible mechanisms for its action are as follows: (1) puromycin entry and peptide bond formation occur only at the termination step and not during elongation or (2) puromycin entry results in fragments too small to be resolved by PAGE techniques. Overall, the assay allows for the testing of efficient peptidyl transferase inhibitors in translation.

A fourth in vitro assay (see Ganoza et al. (2001) Antimicrobial Agents and Chemotherapy 45(10):2813-2819) can be used, for example, to test for inhibition of translation by antibiotics like hygromycin B. Generally, many antibiotics perturb specific ribosomal events and protect bases on the small- and large-subunit rRNAs. Some antibiotics are thought to directly or indirectly disrupt the structure of rRNA. Interestingly, many mutations in rRNA that confer resistance to antibiotics reside in specific bases of either the 16S or 23S rRNA. A known set of antibiotics binds to the 16S rRNA, including streptomycin, neomycin, paromomycin, tetracycline, spectinomycin, and hygromycin B. All these aminoglycoside antibiotics protect bases that are phylogenetically conserved on the 16S rRNA. Further, some of these anitbiotics or derivatices affect the dissociation from ribosomes of the translocase EF-G in its GDP-bound form, however, Hygromycin B, does not affect this specific reaction.

Hygromycin B is thought to have a dual effect on translation by inducing misreading of aminoacyl-tRNAs as well as impairing translocation. Hygromycin is believed to distort the ribosomal A site, thereby inhibiting translation. Furthermore, hygromycin selectively inhibits the ATPase activity of RbbA, a protein that occurs bound to ribosomes and stimulates the ATPase activity of Escherichia coli 70S and 30S ribosomal particles during translation. RbbA accounts for most of the ATPase activity of 70S ribosomes and 30S subunits. This assay shows that the antibiotic hygromycin B can inhibit the 70S ribosome-associated ATPase activity. Specifically, the assay tests if hygromycin B can release the RbbA protein from the ribosomes. Ribosomes are incubated under conditions similar to those used for synthesis with and without the antibiotic, and the particles are removed by ultracentrifugation. The resulting supernatants are analyzed by immunoblots for the presence of RbbA. No RbbA is released from the ribosomes in the absence of antibiotics. Hygromycin B, on the other hand, consistently releases RbbA from the ribosomes. Streptomycin can be used as a control and releases only about 25% of the amount of RbbA that hygromycin B releases. This is consistent with the fact that streptomycin also exhibits about 20 percent inhibition of the ATPase activity of ribosomes compared to that of hygromycin B. Thus, the effect of hygromycin B on the ribosomal ATPase activity is due to its ability to effectively release RbbA from the particles which can be easily tested in this in vitro assay.

Finally a fifth in vitro assay (see Mehta et al. (2003) Curr. Microbiol. 47(3):237-43) tests the ability of aminoglycoside antibiotics to inhibit translation focuses on the inhibition of 30S ribosomal subunit formation. Aminoglycoside antibiotics such as paromomycin and neomycin, are known to bind specifically to the 30S ribosomal subunit. Both antibiotics inhibit translation and protein synthesis. A 3H-uridine pulse and chase assay can be used to examine the kinetics of subunit synthesis in the presence and absence of paromomycin and neomycin. In this assay, the formation of the 30S subunit can be inhibited by both compounds. At 3 μg/mL each antibiotic reduces the rate of 30S formation by about 80 percent compared with control cells. Both paromomycin and neomycin show a concentration-dependent inhibition of particle formation, with a lesser effect on 50S particle formation. For neomycin, the IC50 for 30S particle formation is equal to the IC50 for inhibition of translation. Both antibiotics reduce the viable cell number with an IC50 of 2 μg/mL. Both antibiotics also inhibit protein synthesis in the cells with different IC50 values (2.5 and 1.25 μg/mL). Thus, neomycin and paromomycin are 30S ribosomal subunit-specific antibiotics that prevent assembly of the small subunit. The assay can be used to test any substance that will prevent assembly of the small ribosomal subunit as well as any derivatives, mimetics or analogs of neomycin and paromomycin for their ability to inhibit translation via this mechanism.

2. Culture of Pancreatic Cells with Protein Synthesis Inhibitors

Protein synthesis inhibitors are added to the culture medium of isolated pancreatic cells for selection of pancreatic endocrine lineage cells. Those of skill will recognize that a variety of protein synthesis inhibitors can be used in the methods of the present invention. The protein synthesis inhibitors used are preferably lethal to eukaryotic cells, e.g., the protein synthesis inhibitors cause the death of eukaryotic cells. In some embodiments, the protein synthesis inhibitors are more lethal to dividing eukaryotic cells than to non-dividing eukaryotic cells. In other embodiments, the protein synthesis inhibitors are more lethal to pancreatic exocrine cells than to pancreatic endocrine lineage cells.

Examples of protein synthesis inhibitors that can be used in the invention include macrolides, streptogramins, oxozolidinones, clindamycin, tetracyclines, aminoglycosides and aminonucleosides.

In some embodiments the protein synthesis inhibitors are also antibiotics. Many commercially available antibiotics are protein synthesis inhibitors and can be used in the methods of the invention to select pancreatic endocrine cells. Examples of antibiotics that are protein synthesis inhibitors include e.g., hygromycin, G418 or geniticin sulfate, amikacin, gentamicin, kanamycin, neomycin, netilmicin, paromomycin, streptomycin, and puromycin.

In one preferred embodiment, the protein synthesis inhibitor used to select pancreatic endocrine cells is an aminoglycoside antibiotic. The aminoglycoside antibiotics are related structurally in that they all contain a unique aminocyclitol ring structure. Examples of aminoglycoside antibiotics include, e.g., hygromycin, G418 amikacin, gentamicin, kanamycin, neomycin, netilmicin, paromomycin, and streptomycin.

In another preferred embodiment, the protein synthesis inhibitor is an aminonucleoside antibiotic. Examples of aminonucleoside antibiotics include, e.g., puromycin.

In another preferred embodiment, hygromycin is used to used to select pancreatic endocrine lineage cells.

In still another preferred embodiment, puromycin is used to used to select pancreatic endocrine lineage cells.

In yet another preferred embodiment, G418 is used to used to select pancreatic endocrine lineage cells.

After pancreatic cells are isolated, a protein synthesis inhibitor is added to the culture medium and used to select pancreatic endocrine lineage cells. To select pancreatic endocrine lineage cells from total pancreatic cells, the protein synthesis inhibitor is present in the culture medium in an amount and for a time sufficient to kill many or most pancreatic non-endocrine cells, while permitting survival of pancreatic endocrine lineage cells. The methods of the invention can also be used to select pancreatic endocrine lineage cells from total pancreatic cells by using a protein synthesis inhibitor that is lethal to e.g., pancreatic exocrine cells.

In one embodiment, an inhibitor of protein synthesis is added to a culture medium containing isolated pancreatic cells in an amount that is lethal to at least 30%, 40%, 50%, 60%, 70%, 75%, 80%, 85%, 90%, 91%, 92, 93%, 94%, 95%, 96%, 97%, 98%, or 99% of the cells comprising a culture of isolated pancreatic cells, to allow survival and selection of pancreatic endocrine lineage cells.

In one another embodiment, an inhibitor of protein synthesis is added to a culture medium containing isolated pancreatic cells in an amount that is lethal to at least 30%, 40%, 50%, 60%, 70%, 75%, 80%, 85%, 90%, 91%, 92, 93%, 94%, 95%, 96%, 97%, 98%, or 99% of the pancreatic exocrine cells within the isolated pancreatic cells, to allow survival and selection of pancreatic endocrine lineage cells.

The amount of antibiotic used and the time for exposure that allows selection of pancreatic endocrine lineage cells can be determined according to the needs of the user and according to properties, e.g., solubility or efficacy, of the particular protein synthesis inhibitor. The following ranges of protein synthesis inhibitors are exemplary: 0.1 μg/ml to 2000 μg/ml; 0.1 μg/ml to 1500 μg/ml; 0.5 μg/ml to 1000 μg/ml; 1.0 μg/ml to 100 μg/ml; 2.5 μg/ml to 50 μg/ml. If relatively high concentrations of protein synthesis inhibitors are used, the time of exposure to the protein synthesis inhibitor can be shortened. Incubation times can be from e.g., 2, 5, 6, 7, 8, 9, 10, 11, 12, 13, 14, 15, 16, 17, 18, 19, 20, 21, 22, 23, 24, 28, 32, or 36 hours, or 1, 2, 3, 4, 5, 6, 7, 8, 9, 10, 11, 12, 13, 14, 21, or 30 days. If low concentrations of protein synthesis inhibitors are used, the time of exposure to the protein synthesis inhibitor can be lengthened. Appropriate incubation times and concentrations of protein synthesis inhibitors can be determined by assaying for cell death after application of the protein synthesis inhibitor. Assays disclosed herein can then be used to identify markers in surviving cells that are specific for e.g., pancreatic endocrine lineage cells or pancreatic exocrine cells. After exposure to the protein synthesis inhibitor for an appropriate amount of time, the medium containing the protein synthesis inhibitor is removed and the remaining viable cells, including pancreatic endocrine cells, are washed and allowed to recover. Protein synthesis inhibitor selection can be performed more than once. For example, after a first selection of pancreatic endocrine lineage cells with protein synthesis inhibitors, the surviving cells can be allowed to recover and the protein synthesis inhibitor selection can be performed again.

For example, hygromycin can be used to select pancreatic endocrine lineage cells by its addition to a culture of isolated pancreatic cells for 24 hours at a final concentration of 25 μg/ml, or by its addition to a culture of isolated pancreatic cells for 72 hours at a final concentration of 5 μg/ml, or by its addition to a culture of isolated pancreatic cells for 48 hours at a final concentration of 50 μg/ml, followed by six days of recovery and then addition of 10 μg/ml hygromycin for 24 hours. In another example, puromycin can be used to select pancreatic endocrine lineage cells by its addition to a culture of isolated pancreatic cells for 24 hours at a final concentration of either 2.5 or 5 μg/ml. In a further example, G418 can be used to select pancreatic endocrine lineage cells by its addition to a culture of isolated pancreatic cells for 24-48 hours at a final concentration of either 500 or 1000 μg/ml.

The effectiveness of a protein synthesis inhibitor can be determined being assaying the viability of cells that are incubated with the protein synthesis inhibitor. Death of cells in the presence of the protein synthesis inhibitor indicate the inhibitor is effective. Cell death includes apoptotic and necrotic cell death. Cell death or viability can be assayed using any of a number of techniques available to those of skill in the art. For example, a sample of cells can be removed and assayed for viability using dye exclusion assays. In some instances cell death can be determine by visual inspection, as when dead cells lose attachment to a tissue culture plate or other growth surface or when a majority of total pancreatic cells survive protein synthesis inhibitor selection and the cells reach confluence on the plate.

Death of a particular cell populations, e.g., pancreatic exocrine cells, can be determined by comparing pancreatic exocrine marker levels in a sample of pancreatic cells before addition of protein synthesis inhibitor to a sample of cells that survive protein synthesis inhibitor treatment. Reduction in the levels of e.g., pancreatic exocrine markers, indicates a reduction in the amount of pancreatic exocrine cells in the cell culture.

Protein synthesis inhibitor selection of pancreatic endocrine cells from isolated pancreatic cells can occur at a variety of times after isolation of the pancreatic cells from the donor. In some embodiments, the pancreatic endocrine cells are selected from isolated pancreatic cells using protein synthesis inhibitors before passage of the isolated pancreatic cells, i.e., at passage 0 (P0). However, protein synthesis inhibitor selection of pancreatic endocrine cells from isolated pancreatic cells can also occur at later passages, e.g., P1, P2, P3, P4, P5, and so on.

In some embodiments protein synthesis inhibitors are used to select pancreatic endocrine cells with the caveat the pancreatic endocrine cells are not transfected with an exogenous gene that renders the cells resistant to the presence of the protein synthesis inhibitor in the medium. As an example, exogenous antibiotic resistance genes are well known and used to allow the growth or survival of susceptible cells in the presence of antibiotics, such as e.g., hygromycin and puromycin.

III. Identification and Characterization of Pancreatic Endocrine Cells

Pancreatic endocrine cells are cells that express and secrete hormones, e.g., insulin, into the bloodstream. Thus, one method of identifying pancreatic endocrine cells is to determine levels of insulin mRNA or protein in the cells or levels of insulin, including e.g., C-peptide, secreted by cells.

In some embodiments levels of insulin mRNA in pancreatic endocrine cells are compared to mRNA levels of a housekeeping gene, such as actin. In preferred embodiments, pancreatic endocrine cells express insulin

Levels of other markers can also be determined and used to identify and characterize pancreatic endocrine cells. Intracellular marker include NeuroD1, PDX-1, Glucagon, and CK19. Protein or mRNA levels can be determined in order to characterize pancreatic endocrine cells. Any method used to measure proteins or nucleic acids can be used to determine a level of a marker of a pancreatic endocrine cell. Similarly, measurement of appropriate makers can be used to determine the presence of pancreatic exocrine cells in a cell population.

Pancreatic endocrine cells include progenitor cells that are capable of division and that can differentiate into cells that express high levels of insulin. Pancreatic endocrine cells also include proliferating pancreatic endocrine cells that are actively dividing. Pancreatic endocrine cells also include mature pancreatic endocrine cells that can form insulin producing aggregates or are part of such aggregates and that secrete high levels of insulin.

Those of skill in the art will recognize that it can be useful to determine the differentiation state of CD56 positive cells and their progeny. The differentiation state of pancreatic cells can be determined in a variety of ways, including measurement of protein and mRNA markers of differentiation and functional assays of pancreatic cells, e.g. ability to secrete insulin in response to glucose stimulation.

A. Phenotypic Assays

To know when pancreatic endocrine cells are present, it is useful to assay the phenotypes of pancreatic cells at particular stages of culture. Since expression of particular proteins correlates with cell identity or differentiation state, cells may be analyzed for the expression of a marker gene or protein to assess their identity or differentiation state. For example, in freshly isolated pancreatic tissue, expression of amylase identifies the cell as an exocrine acinar cell, while expression of insulin identifies the cell as an endocrine islet cell. Likewise, islet cells at an early stage of differentiation are usually positive for the cytokeratin CK-19, while mature islet cells show less expression of CK-19.

Phenotypic properties may be assayed on a cell-by-cell basis or as a population average. The mode of assay will depend on the particular requirements and methodology of the assay technique. Thus, assays of marker expression by immunohistochemistry, performed on fixed sections or on suspended cells by FACS analysis, measure the frequency and intensity with which individual cells express a given marker. On the other hand, it may be desirable to measure properties such as the average insulin to actin mRNA expression ratio over an entire population of cells. In such cases, the assay is typically performed by collecting mRNA from a pool of cells and measuring the total abundance of insulin and actin messages. Many phenotypic properties may be assayed either on a cell or population basis. For example, insulin expression may be assayed either by staining individual cells for the presence of insulin in secretory granules, or by lysing a pool of cells and assaying for total insulin protein. Similarly, mRNA abundance may be measured over a population of cells by lysing the cells and collecting the mRNA, or on an individual cell basis by in situ hybridization.

1. Cell Differentiation Markers

There are a number of cellular markers that can be used to identify populations of pancreatic cells. Donor cells isolated and cultured begin to display various phenotypic and genotypic indicia of differentiated pancreatic cells. Examples of the phenotypic and genotypic indicia include various molecular markers present in the facultative progenitor cell population that are modulated (e.g., either up or down regulated). These molecular markers include CK-19, which is hypothesized to be a marker of the pancreatic facultative stem cell.

Typically, mammalian stem cells proceed through a number of developmental stages as they mature to their ultimate developmental endpoint. Developmental stages often can be determined by identifying markers present or absent in developing cells. Because human endocrine cells develop in a similar manner, various markers can be used to identify cells as they transition from a stem cell-like phenotype to pseudoislet phenotype.

The expression of markers in cells induced to proliferate or differentiate by the methods of the present invention bears some similarity to the sequence of marker expression in normal human pancreas development. Very early in development, the primordial epithelial cells express PDX-1, an early cellular marker that is a homeodomain nuclear factor. As the cells develop, they begin to bud out and form a duct. These cells express cytokeratin 19, a marker for epithelial ductal cells, and temporally express PDX-1 leading developmentally to endocrine cells. As these cells continue to develop, they gain the ability to express insulin, somatostatin, or glucagon. The final differentiated cells are only able to express one and become the a cells (glucagon), β cells (insulin), and δ cells (somatostatin). The initial selected pancreatic endocrine cell population used herein is believed to be at a less than fully differentiated stage of development, retaining the ability to proliferate and the potential to differentiate into mature pancreatic endocrine cells. The protein-synthesis-inhibitor-selected pancreatic endocrine cells are able to proliferate as well as to express endocrine hormones and, therefore, have the potential for being used to correct a deficiency in any type of islet cell.

Markers of interest are molecules that are expressed in temporal- and tissue-specific patterns in the pancreas (see Hollingsworth, Ann N Y Acad Sci 880:38-49 (1999)). These molecular markers are divided into three general categories: transcription factors, notch pathway markers, and intermediate filament markers. Examples of transcription factor markers include PDX-1, NeuroD, Nkx-6.1, Isl-1, Pax-6, Pax-4, Ngn-3, and HES-1. Examples of notch pathway markers include Notch1, Notch2, Notch3, Notch4, Jagged1, Jagged2, Dll1, and RBPjk. Examples of intermediate filament markers include CK19 and nestin. Examples of markers of precursors of pancreatic β cells include PDX-1, Pax-4, Ngn-3, and Hb9. Examples of markers of mature pancreatic β cells include insulin, PDX-1, and Hb9. PDX-1, Pax-4, Ngn-3, Hb9, insulin, somatostatin, and glucagon are also used as markers of pancreatic endocrine cells.

Methods for assessing expression of protein and nucleic acid markers in cultured or isolated cells are standard in the art and include quantitative reverse transcription polymerase chain reaction (RT-PCR), Northern blots, and in situ hybridization (see, e.g., Current Protocols in Molecular Biology (Ausubel et al., eds. 2001 supplement)) and immunoassays, such as immunohistochemical analysis of sectioned material, Western blotting, and, for markers that are accessible in intact cells, flow cytometry analysis (FACS) (see, e.g., Harlow and Lane, Using Antibodies: A Laboratory Manual, New York: Cold Spring Harbor Laboratory Press (1998)). Conventional histochemical markers of endocrine cell differentiation may also be employed. Cells to be examined by immunohistochemistry may be cultured on glass chamber slides for microscopic examination. Alternatively, cells grown in conventional tissue culture may be manually removed from the culture and embedded in paraffin for sectioning. PDX-1 antibody can be made following the teachings of Leonard J. et al., Mol. Endocrinol., 1993, Oct. 7, (10) 1275-83.

Cell differentiation markers are varied and can be detected by conventional immunohistochemistry. A generally applicable protocol follows.

The staining process begins with removing chamber portion of the slides. Cells were very gently rinsed with in buffers and fixed in paraformaldehyde solution. Cells are then incubated in a blocking solution containing normal serum at room temperature. Cells were permeabilized with non-ionic detergent in blocking solution. Primary antibodies as listed below are prepared in blocking solution at appropriate dilution and added to cells and incubated. Following incubating with primary antibody, cells were rinsed in buffer and reblocked in blocking solution.

Secondary antibody prepared in blocking solution at appropriate dilution is added to the cells and incubated in the dark. Following incubation the cells are rinsed and nuclei were counterstained with Hoechst dye. Excess fluid is removed and the slides are mounted and covered with coverslides. The slides dry and are stored in the dark.

Alternatively the cells can be prepared for immunocytochemistry using the ABC method. In brief, the cells are embedded in parafin and slides with paraffin sections are dried at 37° C. overnight. The cells are deparaffinized and immersed in a hydrogen peroxide methanol solution to inhibit endogenous peroxidase activity. Slides were boiled in 0.01 citrate buffer (pH 6.0) for 30 minutes to recover certain epitopes. Slides were rinsed with buffer and blocked using normal serum at room temperature in a moist chamber.

Primary antibody prepared in blocking solution are added to the samples and incubated in a moist chamber. Slides are washed and incubated with secondary antibody prepared in blocking solution. Slides were again rinsed with buffer and incubated with Avidin-Horse Reddish Peroxides reagent or ABC complex from a commercial kit (e.g. Dako Corporation). Slides are again rinsed and incubated with diaminobenzidin developing solution; urea hydrogen peroxides in a gold wrap. After washes with distilled water, slides are immersed in Mayer's Hematoxylin for 5 minutes, then kept slides in running tap water until water turned colorless and nuclei were blue. Slides are dehydrated and mounted for viewing.

2. Insulin mRNA Expression

One marker that may be used to characterize pancreatic cell identity, differentiation, or maturity is the level of insulin mRNA. For example, the intermediate cell population of the present invention show expression of insulin mRNA within a defined range. Methods for quantitating insulin mRNA include Northern blots, nuclease protection, and primer extension. In one embodiment, RNA is extracted from a population of cultured cells, and the amount of proinsulin message is measured by quantitative reverse transcription PCR. Following reverse transcription, insulin cDNA is specifically and quantitatively amplified from the sample using primers hybridizing to the insulin cDNA sequence, and amplification conditions under which the amount of amplified product is related to the amount of mRNA present in the sample (see, e.g., Zhou et al, J Biol Chem 272:25648-51 (1997)). Kinetic quantification procedures are preferred due to the accuracy with which starting mRNA levels can be determined.

Frequently, the amount of insulin mRNA is normalized to a constitutively expressed mRNA such as actin, which is specifically amplified from the same RNA sample using actin-specific primers. Thus, the level of expression of insulin mRNA may be reported as the ratio of insulin mRNA amplification products to actin mRNA amplification products, or simply the insulin:actin mRNA ratio. The expression of mRNAs encoding other pancreatic hormones (e.g., somatostatin or glucagon) may be quantitated by the same method. Insulin and actin mRNA levels can also be determined by in situ hybridization and then used to determine insulin:actin mRNA ratios. In situ hybridization methods are known to those of skill in the art. For determination of insulin:actin ratios, either in vitro assays, e.g., amplification, or in istu hybridization methods can be used to quantitiate the messenger RNA. Preferred insulin:actin ratios are e.g., between 0.001 and 10,000; between 0.01 and 1,000; or between 0.1 and 100. The ratios will vary with the developmental stage of the pancreatic endocrine lineage cell. For example, pancreatic endocrine lineage progenitor cells will exhibit lower insulin:actin ratios. In contrast, mature, insulin producing pancreatic endocrine lineage cells will have insulin:actin ratios in the upper ranges.

B. Functional Assays

1. Glucose Stimulated Insulin Secretion

One of the important functions of a beta cell is to adjust its insulin secretion according to the glucose level. Typically, a static glucose stimulation (SGS) assay can be performed on the proliferating adherent pancreatic cells to identify whether they are able to secrete insulin in response to different glucose levels. Cells are generally cultured on an appropriate substrate until nearly confluent. Three days prior to the SGS test, the culture medium is replaced by a medium of similar character but lacking insulin and containing only 1 g/L of glucose. The medium is changed each day for three days and the SGS test is performed on day four.

Before the test, the culture medium may be collected for glucose and insulin analysis. To prepare cells for the test, cells are washed twice with Dulbecco's phosphate-buffered saline (DPBS)+0.5% BSA, incubating for 5 minutes with each wash, and then once with DPBS alone, also incubating for 5 minutes. After washing, the cells are incubated with 10 ml (in a 100 mm dish) or 5 ml (in a 60 mm dish) of Krebs-Ringers SGS solution with 60 mg/dl glucose (KRB-60) for 30 minutes in a 37° C. incubator. This incubation is then repeated.

To perform the SGS assays, cells are incubated in 3 ml (100 mm dish) or 4 ml (T75 flask) or 2 ml (60 mm dish) KRB-60, at 37° C. for 20 minutes. The medium is aspirated and spun, and is collected for insulin assay as LG-1 (low glucose stimulated step). KRB-450+theo (KRB with 450 mg/dl glucose and 10 mM theophylline) is then added with the same volume as above, and cells are cultured under the same condition as above. The supernatant is collected for insulin assay as HG (high glucose stimulated). The cells are then incubated again with KRB-60 and the medium collected as LG-2, and another time as LG-3. The media are collected for insulin analysis, and stored at −20° C. until insulin content is determined by radioimmunoassay (RIA) or other suitable assay.

The results of the SGS test are often expressed as a stimulation index, defined as the HG insulin value divided by the LG-1 insulin value. Generally, a stimulation index of about 2 or greater is considered to be a positive result in the SGS assay, although other values (e.g., 1.5, 2.5, 3.0, 3.5, etc.) may be used to define particular cell populations.

IV. Cell Culture and Cultivation of Pancreatic Endocrine Cells and their Progeny

A. General Cell Culture Procedures

Once the pancreatic cells are obtained and subjected to selection with protein synthesis inhibitors, they are cultured under conditions that select for propagation of the desired pancreatic endocrine cell population, or in other embodiments, for the differentiation of more mature cell types. General cell culture methodology may be found in Freshney, Culture of Animal Cells: A Manual of Basic Technique 4th ed., John Wiley & Sons (2000). Typically, pancreatic endocrine cells are cultured under conditions appropriate to other mammalian cells, e.g., in humidified incubators at 37° C. in an atmosphere of 5% CO₂. Cells may be cultured on a variety of substrates known in the art, e.g., borosilicate glass tubes, bottles, dishes, cloning rings with negative surface charge, plastic tissue culture tubes, dishes, flasks, multi-well plates, containers with increased growth surface area (GSA) or Esophageal Doppler Monitor (EDM) finish, flasks with multiple internal sheets to increase GSA, Fenwal bags, and other culture containers.

Once the pancreatic cellular material has been harvested and selected for culture, or once a population is confluent and is to be transferred to a new substrate, a population of cells is seeded to a suitable tissue culture container for cultivation. Seeding densities can have an effect on the viability of the pancreatic cells cultured using the disclosed methods, and optimal seeding densities for a particular culture condition may be determined empirically by seeding the cells at a range of different densities and monitoring the resulting cell survival and proliferation rate. A range of seeding densities has been shown to be effective in producing hormone secreting cells in culture. Typically, cell concentrations range from about 10² to 10⁸ cells per 100 mm culture dish, e.g., 10², 10³, 10⁴, 10⁵, 10⁶, 10⁷, or 10⁸ cells per 100 mm culture dish, although lower cell concentrations may be employed for cloning procedures. Cell concentration for other culture vessels may be adjusted by computing the relative substrate surface area and/or medium gas exchange surface area for a different culture vessel. For example, a typical 100 mm culture dish has a substrate surface area of 55 square centimeters (see Freshney, supra), and a cell concentration of 10,000 cells per dish corresponds to about 180 cells per square centimeter, while a cell concentration of 100,000 cells per dish corresponds to about 1,800 cells per square centimeter. Cell concentration in terms of culture vessel surface area may be related to cell concentration in terms of media volume by using the appropriate media volume per culture surface area (0.2-0.5 ml/cm² are typical ranges for static culture). To determine if a 10 fold expansion has occurred, the cells are removed by enzymatic digestion and counted under microscope in a known volume of fluid. Cells may also be grown on culture surfaces pre-coated with defined extracellular matrix components to encourage growth and differentiation (e.g., fibronectin, Collagen I, Engelbreth-Holm-Swarm matrix, and, preferably, collagen IV or laminin).

Standard cell culture propagation techniques are suitable for practice of the invention. When cells are growing attached to a culture surface, they are typically grown as a monolayer until 80%-90% confluence is reached, at which point the cells are released from the surface by proteolytic digestion and split 1:2 or 1:3 for culture in new vessels. Higher dilutions of the cells are also suitable, generally between the ranges of 1:4 to 1:10, although even lower cell concentrations are appropriate in cloning procedures. Concentrations of proteolytic enzymes and chelating agents are usually lowered when cells are passaged in serum-free media (e.g., 0.025% trypsin and 0.53 mM EDTA). Culture medium is typically changed twice weekly or when the pH of the medium indicates that fresh medium is needed.

The pancreatic cells of the present invention may be cultured in a variety of media. As described herein, media containing or lacking particular components, especially serum, are preferred for certain steps of the isolation and propagation procedures. For example, cells freshly isolated from the pancreas may be maintained in high serum medium to allow the cells to recover from the isolation procedure. Conversely, low serum medium favors the selection and propagation of an intermediate stage population of pancreatic endocrine cells. Accordingly, a number of media formulations are useful in the practice of the invention. The media formulations disclosed here are for exemplary purposes, and non-critical components of the media may be omitted, substituted, varied, or added to simply by assaying the effect of the variation on the replication or differentiation of the cell population, using the assays described herein. See, e.g., Stephan et al., Endocrinology 140:5841-54 (1999)).

Culture media usually comprise a basal medium, which includes inorganic salts, buffers, amino acids, vitamins, an energy source, and, in some cases, additional nutrients in the form of organic intermediates and precursors that are involved in protein, nucleic acid, carbohydrate, or lipid metabolism. Basal media include F12, Eagle's MEM, Dulbecco's modified MEM (DMEM), RPMI 1640, a 1:1 mixture of F12 and DMEM, and others. See Freshney, supra. To support the growth of cells, basal medium is usually supplemented with a source of growth factors, other proteins, hormones, and trace elements. These supplements encourage growth, maintenance, and/or differentiation of cells, compensate for impurities or toxins in other medium components, and provide micronutrients lacking in the basal medium. In many culture media, serum is the source of these supplements. Serum can be supplied from a variety of mammalian sources, such as human, bovine, ovine, equine, and the like, and from adult, juvenile, or fetal sources. See Freshney, supra. Fetal bovine serum is a commonly used supplement. Concentrations of serum are expressed in terms of volume of serum as a percentage of the total medium volume, and typically range from about 0.1 to 25%, e.g., about 0.1, 0.2, 0.5, 1, 2, 3, 4, 5, 6, 7, 8, 9, 10, 15, 20, or 25%. In some applications, the basal medium is supplemented with defined or semi-defined mixtures of growth factors, hormones, and micronutrients, rather than with serum. Formulas for serum replacement supplements are disclosed herein; others are known in the art or available from commercial sources (see Freshney, supra). For some embodiments, the concentration of serum is lowered but not eliminated, and defined or semi-defined supplement mixtures are added to the basal medium. Preferred applications for media containing high or low concentrations of serum are described herein.

B. Maintenance and Propagation of Isolated Pancreatic Cells in Media Containing High Serum

Cells harvested from a donor pancreas have usually undergone a period of warm or cold ischemia between the death of the donor and the beginning of the isolation procedure. Moreover, during the isolation procedure, pancreatic cells are usually subjected to proteolytic digestion as well as mechanical and shear stresses. Without wishing to be bound by a particular theory, the various traumas experienced by these cells may up-regulate various cellular processes that result in the expansion of pancreatic stem cell populations, such as facultative progenitor cells. Intermediate cell populations may be generated with satisfactory efficiency by placing cells into low serum media directly after isolation or purification. Nonetheless, because the trauma experienced by cells during the isolation procedures may have adverse effects on cell survival and adaptation to culture, it is sometimes desirable to maintain the freshly isolated cells in a stabilizing medium containing high concentrations of serum (e.g., >4%) to improve the efficiency of the culturing process. This maintenance period may be brief (e.g., overnight). Optionally, cells may be maintained for an extended propagation period in high serum medium.

High serum media for stabilization will typically contain at least 4% serum, and, in some embodiments, will contain a higher concentration of serum such as 10% or 20%. Media used for stabilization or propagation may be derived from a basal medium such as RPMI 1640, available from many commercial sources and described by Moore et al., J Am Med Assoc 199:519-524 (1967)). Exemplary high serum media for maintenance or propagation include Medium 3 (RPMI 1640+10 mM HEPES, 2 mM glutamine, 5 M ZnSO₄, and 10% fetal bovine serum (FBS)) and Medium 7 (RPMI 1640+10 mM HEPES, 2 mM glutamine, 5 μM ZnSO₄, and 20% FBS). High serum media may also be derived by mixing a particular volume of high serum medium such as Medium 3 or Medium 7 with a particular volume of serum-free medium such as SM95, SM96, or SM98 (described herein) to arrive at a desired serum concentration (e.g., 4%-9%).

For stabilization after harvest, cells are conveniently cultured in a culture vessel at relatively high densities in a high serum medium (e.g., 10⁹ cells in 70 ml of Medium 7 (20% FBS)). However, lower cell densities and serum concentrations may be employed as well. Cells are typically maintained in the original vessel for a relatively short time (e.g., overnight) to allow for recovery from the harvesting procedure.

Following the maintenance period, cells may be transferred to low serum media for selection and propagation of the pancreatic endocrine cell population as described herein. Optionally, the cells may be cultured in a high serum medium to allow for proliferation of the mixed cell population. In a typical embodiment, cells from the maintenance culture are reseeded into a new culture vessel containing Medium 3 (10% FBS), Medium 7 (20% FBS), or a mixture of Medium 3 and Medium 7 (15% FBS), or other AmCyte culture media. Cells are typically cultured in this medium for 7-10 days, during which time they may grow to confluence. Once the cells have reached confluence, they may be passaged into low serum media for selective expansion of the intermediate cell population described herein.

C. Expansion and Propagation of a Pancreatic Endocrine Cell Population by Culture in Media Containing Low Serum

Once the pancreatic endocrine cells have been isolated, the cells are then transferred to a selective medium to promote propagation of the pancreatic endocrine cell population. This selective medium favors propagation of cells which retain the ability to secrete pancreatic endocrine hormones, or which retain the potential to mature into more differentiated cells which secrete high levels of pancreatic endocrine hormones. In general, selective medium will favor propagation of epithelial or epithelial-like cells at the expense of fibroblasts and mesenchymal cells, although pure epithelial cultures have not been shown to be required for the advantageous use of pancreatic cells in the methods of the invention. Typically, epithelial-selective media will yield a population of nearly pure (e.g., <10% fibroblasts or mesenchymal cells) cells after a certain period of growth in culture, e.g., 2, 3, 4, or 5 passages depending on the expansion of the population in each passage.

One type of selective medium which has been employed to favor epithelial cell growth from embryonic tissues is serum-free medium (see, e.g., Stephan et al., supra; Peehl and Ham, In Vitro 16:526-40 (1980)). Epithelial-specific media, and, more preferably, low serum media containing a source of growth hormone, may be employed to select for a distinct population of propagating pancreatic cells from adult mammals that retain markers of pancreatic cell development (e.g., PDX-1), but can be further differentiated under appropriate conditions to express high levels of pancreatic endocrine hormones. Particular epithelial-selective media suitable for culture of pancreatic cells are disclosed herein, but other medium formulations known in the art to favor the preferential expansion of epithelial or epithelial-like cells may also be employed.

The transfer to epithelial-selective low serum medium may be accomplished after a period of maintenance in high serum medium (“weaning”), or by transferring the cells directly into selective low serum medium following the isolation and separation procedure (“shock”). Either methodology is suitable for generation of the desired intermediate cell population.

1. Growth Hormones and Preferred Examples

Epithelial selective culture media containing growth hormone (GH) is used promote the emergence of a valuable pancreatic cell population of intermediate differentiation. Without wishing to be bound by a particular theory, it is hypothesized that GH can replace the mitogenic substances ordinarily found in serum that support cell growth, but that serum contains other mitogenic factors that promote the overgrowth of less desirable cell populations (e.g., fibroblasts and mesenchymal cells). Hence, replacement of serum with a supplemental mixture containing GH selects for propagation of a cell population with an intermediate state of differentiation. While the functions of GH in serum-free medium may be substituted with other supplemental ingredients in alternative embodiments of the invention, the ready availability of GH in natural extracts or as recombinant protein makes GH-containing media suitable epithelial-selective media for the methods disclosed herein.

Growth hormones, also known as somatotropins, are polypeptide hormones synthesized in the anterior pituitary which promote normal body growth and lactation and influence various aspects of cellular metabolism. GH has both direct effects on cells and indirect effects mediated by IGF-I and similar molecules; in the intact pancreas, islet cell growth has been connected to the expression of GH and the homologous hormones prolactin and lactogen (see, e.g., Nielsen et al., J Mol Med 77(1):62-6 (1999). In humans, mature GH contains 191 amino acid residues and displays a molecular mass of 22 kDa. However, in addition to the commonly observed disulfide dimer, two peptides made of portions of human GH (residues 1-43 and 44-191) have been detected in serum and have distinct effects on adult islet tissue (see Lewis et al., Endocr J 47 Suppl:S1-8 (2000)). Various naturally occurring derivatives, variants, metabolic products, and engineered derivatives of human GH are known, including glycosylated GH, methionyl GH, 20 kDa GH, acetylated GH, proteolytically cleaved GH, desamido GH, sulfoxide GH, and truncated forms of GH.

GH is a member of a conserved family of hormones including, in humans, GH-V1 and GH-V2, choriomammotropin and prolactin and proteins from other vertebrates such as rodent placental lactogens I and II and other bovine and sheep lactogens, murine proliferin I, II, and III and proliferin-related protein, bovine prolactin-related proteins I, II, and III, rat prolactin-like proteins A and B, and somatolactins from various fishes. Members of this family are characterized by the consensus sequences C-x-[ST]-x(2)-[LIVMFY]-x-[LIVMSTA]-P-x(5)-[TALIV]-x(7)-[LIVMFY]-x(6)-[LIVMFY]-x(2)-[STA]-W or C-[LIVMFY]-x(2)-D-[LIVMFYSTA]-x(5)-[LIVMFY]-x(2)-[LIVMFYT]-x(2)-C.

Growth hormone suitable for practice of the invention may be obtained from a variety of natural and artificial sources. In contrast to therapeutic uses of GH, which often require GH of the same species, GH from a range of primate, mammalian, or vertebrate species may be employed in formulation of low serum media for culture of pancreatic cells. A convenient source of growth hormone is bovine pituitary extract (BPE), which is a rich source of natural GH. BPE (75 μg/ml protein) may be included in the culture medium at about 0.1 to 100 μl/ml, preferably at 0.5 to 50 μl/ml, and most preferably at 5 μl/ml or 37.5 mg/l. Pituitary extracts available from other species (e.g., porcine, ovine, and the like) may also be employed at similar concentrations. Other factors present in pituitary extract may potentiate its effect, but satisfactory results may also be achieved with purified GH, and with recombinant GH. Recombinant bovine and human GH are widely available and are a suitable source of GH activity. Recombinant GH may be added to culture medium at between 0.01 and 100 mg/l, preferably between 0.1 and 10 mg/l, more preferably at about 0.2, 0.5, 0.75, 1, 1.25, 2, or 5 mg/l, and most preferably at about 1.25 mg/L, where 1 mg of recombinant protein is about equivalent to 3 IU of GH.

2. Other Supplements

Typical ingredients added to basal media for complete serum-free media include recombinant human insulin (0.1 to 100 μg/ml), transferrin (0.1 to 100 μg/ml), epidermal growth factor (0.1 to 100 ng/ml), ethanolamine (0.1 to 100 μg/ml), aprotinin (0.1 to 100 μg/ml), glucose (0.1 to 100 mg/ml), phosphoethanolamine (0.1 to 100 μM), triiodothyronone (0.1 to 100 μM), selenium (0.1 to 100 nM), hydrocortisone (0.01 to 100M), progesterone (0.1 to 10 nM), forskolin (0.1 to 100 μM), heregulin (0.1 to 100 nM), and bovine pituitary extract (0.1 to 500 μg/ml). Not all supplemental ingredients are required to support cell growth; the optimal concentration or necessity for a particular supplement may be determined empirically, by leaving out or reducing the concentration of a single ingredient and observing the effect on cell proliferation. See e.g., Stephan et al., supra.

In general, supplemental ingredients may be replaced by natural or synthetic products that have the same biological properties. For example, triiodothyronone, hydrocortisone, and progesterone may all be replaced by natural or synthetic hormones known to activate the same intracellular receptors (thyroid receptors, glucocorticoid receptors, and progesterone receptors). Insulin and EGF are typically human proteins produced by recombinant DNA methodology, but may be replaced by polypeptides purified from natural sources, by polypeptides from other species, or by other agonists of the insulin and EGF receptors. GH may, in some cases, be substituted with other antagonists of the GH receptor. Likewise, heregulin, a ligand of the ErbB3 receptor, may be replaced by heregulin isoforms and other ErbB3 agonists such as NRG2, NRG3, and NRG4, sensory and motor neuron-derived factor, neurestin, and Ebp-1, heregulin α, heregulin β, heregulin γ, neuregulin-1 and neuregulin-2 (NRG-1 alpha, NRG-1beta, NRG-2 alpha, and NRG-2 beta.

Exemplary serum-free media include the basal medium SM96 and the complete medium SM95, which consists of SM96 supplemented as shown in the following tables. SM98 consists of 1:1 F12/DMEM supplemented with a modification of medium supplement 14 F described by Stephan et al., supra. SM98 contains less heregulin (1 ng/ml v. 8 ng/ml) than 14 F. Thus, SM 98 consists of 1:1 F12/DMEM supplemented with recombinant human insulin, 10 μg/ml; transferrin, 10 μg/ml; epidermal growth factor, 10 ng/ml; ethanolamine, 61 ng/ml; aprotinin, 25 μg/ml; glucose, 5 mg/ml; phosphoethanolamine, 141 ng/ml; triiodothyronone, 3.365 μg/ml; selenium, 4.325 ng/ml; hydrocortisone, 181 ng/ml; progesterone, 3.15 ng/ml; forskolin, 410 ng/ml; heregulin, 1 ng/ml; and bovine pituitary extract, 75 μg/ml. Exemplary sources of EGH and heregulin in SM95 and SM98 are recombinant human EGF (Sigma E9644) and the EGF domain (amino acids 176-246) of human heregulin-β1 (R&D systems 396-HB/CF).

### RPMI 1640 Media

### (Moore, et al., A.M.A., 199:519 (1967))

| | Mg/L |
|---|---|
| INORGANIC SALTS | |
| Ca(NO₃)₂-4H₂O | 100 |
| KCl | 400.00 |
| MgSO₄ (anhyd.) | 48.84 |
| NaCl | 5850.00 |
| Na₂HPO₄ (anhyd.) | 800.00 |
| OTHER COMPONENTS | |
| D-Glucose | 2000.00 |
| Glutathione (reduced) | 1.0 |
| HEPES | 5958.00 |
| Phenol Red | 5.00 |
| AMINO ACIDS | |
| L-Arginine | 200.00 |
| L-Asparagine (free base) | 50.00 |
| L-Aspartic Acid | 20.00 |
| L-Cystine.2HCl | 65.00 |
| L-Glutamic Acid | 20.00 |
| L-Glutamine | 300.00 |
| Glycine | 10.00 |
| L-Histidine (free base) | 15.00 |
| L-Isoleucine | 50.00 |
| L-Leucine | 50.00 |
| L-Lysine.HCl | 40.00 |
| L-Methionine | 15.00 |
| L-Phenylalanine | 15.00 |
| L-Proline | 20.00 |
| L-Serine | 30.00 |
| L-Threonine | 20.00 |
| L-Tryptophan | 5.00 |
| L-Tyrosine.2Na₂H₂0 | 29.00 |
| L-Valine | 20.00 |
| VITAMINS | |
| Biotin | 0.20 |
| D-Ca Pantothenate | 0.25 |
| Choline Chloride | 3.00 |
| Folic Acid | 1.00 |
| i-Inositol | 35.00 |
| Niacinamide | 1.00 |
| Pyridoxine.HCl | 1.00 |
| Riboflavin | 0.20 |
| Thiamine.HCl | 1.00 |
| Thymidine | 0.005 |
| Vitamin B₁₂ | 1.04 |

SM95

| | |
|---|---|
| INORGANIC SALTS | |
| CaCl₂ | 78.3 |
| CuS0₄.5H₂0 | 0.00165 |
| Fe(NO₃)₃.9H₂O | 0.025 |
| FeSO₄.7H₂0 | 0.61 |
| KCl | 271 |
| MgCl₂ | 28.36 |
| MgSO₄ | 39.06 |
| KH₂PO₄ | 34 |
| NaCl | 7262.75 |
| NaHCO₃ | 1600 |
| Na₂HPO₄ | 101.5 |
| NaH₂PO₄.H₂O | 31.25 |
| ZnS0₄.7H₂O | 0.416 |
| AMINO ACIDS | |
| L-Alanine | 11.225 |
| L-Arginine.HCl | 283.75 |
| L-Asparagine.H₂0 | 18.75 |
| L-Aspartic Acid | 16.325 |
| L-Cysteine.H₂0(non-animal) | 43.78 |
| L-Cystine.2HCl | 15.65 |
| L-Glutamic Acid | 18.675 |
| L-Glutamax I | 328.5 |
| Glycine | 89.375 |
| Glycyl-Histidyl-Lysine | 0.000005 |
| L-Histidine HCl.H₂0 | 38.69 |
| L-Isoleucine | 31.24 |
| L-Leucine | 42.5 |
| L-Lysine.HCl | 82.125 |
| L-Methionine | 13.12 |
| L-Phenylalanine | 22.74 |
| L-Proline | 43.625 |
| L-Serine | 23.625 |
| L-Threonine | 38.726 |
| L-Tryptophan | 6.51 |
| L-Tyrosine.2Na₂H₂0 (non-animal) | 35.9 |
| L-Valine | 38.125 |
| OTHER COMPONENTS | |
| D-Glucose | 3000 |
| HEPES | 1787.25 |
| Na Hypoxanthine | 3.2 |
| Linoleic Acid | 0.066 |
| Lipoic Acid | 0.1525 |
| Phenol Red | 4.675 |
| Na Putrescine.2HCl | 0.191 |
| Na Pyruvate | 137.5 |
| VITAMINS | |
| Biotin | 0.037 |
| Ascorbic Acid | 22.5 |
| D-Ca Pantothenate | 1.37 |
| Choline Chloride | 11.49 |
| Folic Acid | 1.826 |
| L-Inositol | 24.3 |
| Niacinamide | 1.03 |
| Pyridoxine.HCl | 1.046 |
| Riboflavin | 0.13 |
| Thiamine.HCl | 1.23 |
| Thymidine | 0.5325 |
| Vitamin B₁₂ | 1.04 |
| SUPPLEMENTS | |
| Na Selenous Acid | 0.0034 |
| Epithelial Growth Factor | 0.005 |
| Ethanolamine | 0.03 |
| Phosphoethanolamine | 0.07 |
| Aprotinin | 12.5 |
| Progesterone | 0.0016 |
| Forskolin | 0.205 |
| HeregulinB | 0.004 |
| Bovine Pituitary Extract | 37.5 |
| Hydrocortisone | 0.0923 |
| r.h. insulin | 5.05 |
| T₃ | 0.0000015 |
| L-Thyroxine Na | 0.00002 |
| Bovine Transferrin APG | 7.5 |

SM96

| | |
|---|---|
| INORGANIC SALTS | |
| CaCl₂ | 78.3 |
| CuS0₄.5H₂0 | 0.00165 |
| Fe(NO₃)₃.9H₂O | 0.025 |
| FeSO₄.7H₂0 | 0.61 |
| KCl | 271 |
| MgCl₂ | 28.36 |
| MgSO₄ | 39.06 |
| KH₂PO₄ | 34 |
| NaCl | 7262.75 |
| NaHCO₃ | 1600 |
| Na₂HPO₄ | 101.5 |
| NaH₂PO₄.H₂O | 31.25 |
| ZnS0₄.7H₂O | 0.416 |
| AMINO ACIDS | |
| L-Alanine | 11.225 |
| L-Arginine.HCl | 283.75 |
| L-Asparagine.H₂0 | 18.75 |
| L-Aspartic Acid | 16.325 |
| L-Cysteine.H₂0(non-animal) | 43.78 |
| L-Cystine.2HCl | 15.65 |
| L-Glutamic Acid | 18.675 |
| L-Glutamax I | 328.5 |
| Glycine | 89.375 |
| Glycyl-Histidyl-Lysine | 0.000005 |
| L-Histidine HCl.H₂0 | 38.69 |
| L-Isoleucine | 31.24 |
| L-Leucine | 42.5 |
| L-Lysine.HCl | 82.125 |
| L-Methionine | 13.12 |
| L-Phenylalanine | 22.74 |
| L-Proline | 43.625 |
| L-Serine | 23.625 |
| L-Threonine | 38.726 |
| L-Tryptophan | 6.51 |
| L-Tyrosine.2Na₂H₂0 (non-animal) | 35.9 |
| L-Valine | 38.1261 |
| OTHER COMPONENTS | |
| D-Glucose | 3000 |
| HEPES | 1787.25 |
| Na Hypoxanthine | 3.2 |
| Linoleic Acid | 0.066 |
| Lipoic Acid | 0.1525 |
| Phenol Red | 4.675 |
| Na Putrescine.2HCl | 0.191 |
| Na Pyruvate | 137.5 |
| VITAMINS | |
| Biotin | 0.037 |
| Ascorbic Acid | 22.5 |
| D-Ca Pantothenate | 1.37 |
| Choline Chloride | 11.49 |
| Folic Acid | 1.826 |
| i-Inositol | 24.3 |
| Niacinamide | 1.03 |
| Pyridoxine.HCl | 1.046 |
| Riboflavin | 0.13 |
| Thiamine.HCl | 1.23 |
| Thymidine | 0.6325 |
| Vitamin B₁₂ | 1.04 |

### 3. Transfer of Cells to Low Serum Media

Transferring a culture of pancreatic endocrine cells to low serum media promotes the selection of a defined population of cells with an intermediate state of differentiation. This cell population will continue to proliferate if subcultured, but maintains high expression levels of pancreatic markers such as PDX-1. Unstimulated, this population secretes relatively low levels of pancreatic endocrine hormones such as insulin, but can be matured according to the methods of the invention to yield high-secreting cells. To transfer a culture of pancreatic endocrine cells to low serum medium, the cells may be weaned from high serum to low serum media, or may be placed directly in low serum media following isolation. Medium such as SM95 and SM98 are suitable low serum media, although SM95 yields slightly improved insulin secretion upon maturation the of pancreatic cells.

The pancreatic endocrine cell population and its progeny typically retains both the ability to proliferate and the ability for further differentiation into high-secreting endocrine cells. As the pancreatic endocrine cells proliferate, the strength of some marker expression, e.g., CD56, can become less pronounced, and in some cases is detectable only by RT-PCR.

The ability of protein synthesis inhibitor-selected pancreatic endocrine cells to proliferate provides an advantage in their ability to expand and increase the number of cells available for later maturation into glucose-secreting, insulin-producing aggregates. Proliferative ability is generally assessed by the ability of a culture seeded at a one density to expand to a second density; e.g., cells plated at 180 cells per square centimeter may be expanded to 1,800 cells per ml in a single passage. By repeated cycles of propagation and passage, a starting population of isolated pancreatic cells may be expanded by about 10,000-fold or more (e.g., about 100-fold, 500-fold, 1000-fold, 5000-fold, 10,000-fold, 50,000-fold, 100,000-fold, 500,000-fold, or 1,000,000 fold) while retaining endocrine markers such as PDX-1 and insulin mRNA expression, and retaining the ability to differentiate into mature high-secreting endocrine cells.

V. Differentiation-Induction of Insulin Producing Aggregates

Cell differentiation of protein-synthesis-inhibitor-selected pancreatic endocrine cells can be induced through induction of cell aggregation. As the pancreatic endocrine cells differentiate, the strength of expression of certain markers, e.g., CD56 expression, can become less pronounced. Cell aggregation can be induced in a variety of ways. For example, aggregation and differentiation can be induced by growing the cells to confluence. Aggregation and differentiation can also be induced by growing cells on conditioned culture dishes.

A variety of substrates can be used to condition culture dishes. Conditioned culture dishes can be culture dishes that have been used previously to grow intermediate stage pancreatic stem cells. Once the cells have formed a monolayer (typically about 5 days, depending on the initial subculture seeding density), they are removed by trypsinization. Growth of a 100% confluent cell culture is not required to produce a conditioned culture dish. A lowered concentration of trypsin (typically ½ or ¼ of the concentration employed in standard cell culture techniques) is preferred to prevent extensive degradation of the matrix. Alternatively, the cell monolayer may be removed by extracting the substrate with detergent, which will remove the cells but leave behind the secreted matrix (see Gospodarowicz et al, Proc Natl Acad Sci USA 77:4094-8 (1980)).

Conveniently, the removed cells which previously grew on the substrate or culture dish may be split and reseeded on the same, now conditioned, culture dish. However, the culture which conditions the substrate and the culture which is seeded on the substrate need not be the same culture. Accordingly, one culture of cells may be grown on a substrate to condition the substrate, the cells removed, and cells from another culture seeded upon the conditioned substrate. The conditioning cells may be from the same or different donor or species as the cells subsequently cultured.

In another embodiment, plates conditioned with collagen coating are used in the invention. Collagen coated plates are commercially available. In a preferred embodiment, collagen IV coated plates are used to induce aggregation and differentiation of pancreatic cells.

Differentiation of protein-synthesis-inhibitor-selected pancreatic endocrine cells into mature insulin producing cells can also be enhanced by growth of the cells in the presence of differentiation factors. Preferred differentiation factors include hepatocyte growth factor, keratinocyte growth factor, and exendin-4. Hepatocyte growth factor has been shown to effect differentiation of pancreatic cells in culture and in transgenic animals. See e.g., Mashima, H. et al., Endocrinology, 137:3969-3976 (1996); Garcia-Ocana, A. et al., J. Biol. Chem. 275:1226-1232 (2000); and Gahr, S. et al., J. Mol. Endocrinol. 28:99-110 (2002). Keratinocyte growth factor has been shown to effect differentiation of pancreatic cells in transgenic animals. See e.g., Krakowski, M. L., et al., Am. J. Path. 154:683-691 (1999) and Krakowski, M. L., et al., J. Endochrinol. 162:167-175 (1999). Exendin-4 has been shown to effect differentiation of pancreatic cells in culture. See e.g., Doyle M. E. and Egan J. M., Recent Prog. Horm. Res. 56:377-399 (2001) and Goke, R., et al., J. Biol. Chem. 268:19650-19655 (1993). bFGF has been shown to increase the insulin secretion in microencapsulated pancreatic islets. See e.g., Wang W., et al., Cell Transplant 10(4-5): 465-471 (2001). IGF-I has an effect on differentiation of pancreatic ductal cells and IGF-I replacement therapy has been used for type I diabetes treatment. See e.g., Smith F E., et al., Proc. Natl. Acad. Sci. USA. 15; 88(14): 6152-6156 (1991), Thrailkill K M. et al., Diabetes Technol. Ther. 2(1): 69-80 (2000). Evidence has shown that NGF plays an important autoregulatory role in pancreatic beta-cell function. See e.g. Rosenbaum T. et al., Diabetes 50(8): 1755-1762 (2001), Vidaltamayo R. et al., FASEB 16(8): 891-892 (2002), and Pierucci D. et al., Diabetologia 44(10): 1281-1295 (2001). EGF has been shown to promote islet growth and stimulate insulin secretion. See e.g., Chatterjee A K. et al., Horm. Metab. Res. 18(12): 873-874 (1986). PDGF has been shown to have an effect on the survival of CD56-positive cells. See e.g., Ben-Hur T. et al., J. Neurosci. 18(15): 5777-5788 (1998).

VI. Implantation of Pancreatic Endocrine Cells or Progeny and Restoration of Pancreatic Endocrine Function

Those of skill in the art will recognize that propagating pancreatic endocrine cells provide a renewable resource for implantation and restoration of pancreatic function in a mammal. Propagating pancreatic endocrine pancreatic cells are first differentiated before implantation into the mammal. If desired by the user, pancreatic endocrine cells can be encapsulated before implantation.

A. Encapsulation

Encapsulation of the pancreatic endocrine cells results in the formation of cellular aggregates in the capsules. Encapsulation can allow the pancreatic cells to be transplanted into a diabetic host, while minimizing the immune response of the host animal. The porosity of the encapsulation membrane can be selected to allow secretion of biomaterials, like insulin, from the capsule, while limiting access of the host's immune system to the foreign cells.

Encapsulation methods are known in the art and are disclosed in the following references: van Schelfgaarde & de Vos, J. Mol. Med. 77:199-205 (1999), Uludag et al. Adv. Drug Del Rev. 42:29-64 (2000) and U.S. Pat. Nos. 5,762,959, 5,550,178, and 5,578,314. Below is a general description of encapsulation of intermediate stage pancreatic stem cells. Specific examples are found in Examples 5 and 9 of this application.

Encapsulation methods are described in detail in co-pending application PCT/US02/41616; herein incorporated by reference.

B. Implantation

Implantation or transplantation into a mammal and subsequent monitoring of endocrine function may be carried out according to methods commonly employed for islet transplantation; see, e.g., Ryan et al, Diabetes 50:710-19 (2001); Peck et al., Ann Med 33:186-92 (2001); Shapiro et al., N Engl J Med 343(4):230-8 (2000); Carlsson et al., Ups J Med Sci 105(2):107-23 (2000) and Kuhtreiber, W M, Cell Encapsulation Technology and Therapeutics, Birkhauser, Boston, 1999. Preferred sites of implantation include the peritoneal cavity, the liver, and the kidney capsule.

One of skill in the art will be able to determine an appropriate dosage of microcapsules for an intended recipient. The dosage will depend on the insulin requirements of the recipient. Insulin levels secreted by the microcapsules can be determined immunologically or by amount of biological activity. The recipients body weight can also be taken into account when determining the dosage. If necessary, more than one implantation can be performed as the recipient's response to the encapsulated cells is monitored. Thus, the response to implantation can be used as a guide for the dosage of encapsulated cells. (Ryan et al., Diabetes 50:710-19 (2001))

C. In Vivo Measure of Pancreatic Endocrine Function

The function of encapsulated cells in a recipient can be determined by monitoring the response of the recipient to glucose. Implantation of the encapsulated cells can result in control of blood glucose levels. In addition, evidence of increased levels of pancreatic endocrine hormones, insulin, C-peptide, glucagon, and somatostatin can indicate function of the transplanted encapsulated cells.

One of skill in the art will recognize that control of blood glucose can be monitored in different ways. For example, blood glucose can be measured directly, as can body weight and insulin requirements. Oral glucose tolerance tests can also be given. Renal function can also be determined as can other metabolic parameters. (Soon-Shiong, P. et al., PNAS USA 90:5843-5847 (1993); Soon-Shiong, P. et al., Lancet 343:950-951 (1994)).

It must be noted that as used herein and in the appended claims, the singular forms “a”, “and”, and “the” include plural referents unless the context clearly dictates otherwise. Thus, for example, reference to “a cell” includes a plurality of such cells and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. Citations are incorporated herein by reference.

### EXAMPLES

### Example 1

### Selection for Pancreatic Endocrine Cells Using Hygromycin B

Pancreatic cells were isolated from donor pancreas as described in published US Patent Application No. 20040115805, which is herein incorporated by reference for all purposes.

Samples of HD437 P0 pancreatic primary cells from prepurified (PP) layer were cultured in chamber slides in M3 media for 2 days. Hygromycin (25 μg/ml) was then added to the medium for 24 hours. The cells were given same treatment after then cultured for six days in medium M3+ (M3 with supplements containing ITS (1:100), leukemia inhibitory factor (LIF) (1000 u/ml), and alphidicolin (2 μg/ml)) for recovery and synchronization and then the hygromycin B treatment was repeated. Three days after the second treatment one set of cells was fixed for immunocytochemistry analysis and the other set was harvested and analyzed by quantitative RT-PCR. A control group received no hygromycin B and was cultured in M3 for 6 days and had to be harvested for analyses because cells reached confluence. Staining of cells with CD56-specific antibody showed that both CD56 positive and negative cells are present in cultured primary pancreatic cells. However, cells that survived after hygromycin B selection expressed CD56. Cells that survived hygromycin B selection grew in tight patches with a dense core in the center in most cases. Although alphidicolin, a diterpene fungal metabolite that blocks cell cycle at early S-phase, was added to the culture medium after treatment, some proliferation of cells post hygromycin B treatment from those patches was observed, as evident by the increased in cell patch size. The experiment was repeated using HD438 P0 pancreatic primary cells from islet (I) layer and same results were seen.

### Example 2

### Characterization of Hygromycin B Selected Pancreatic Endocrine Cells

Expression of NeuroD1, a pancreatic progenitor marker was determined by quantitative RT-PCR in P0 cells from HD437PP and HD438I with or without hygromycin B treatment. The NeuroD1 mRNA was normalized to β-actin mRNA and the expression levels are indicated by the ratio of mRNA of NeuroD1 to β-actin. Table 1 shows the results. Cells that survived after hygromycin B treatment expressed more NeuroD1 especially cells from HD437PP. The cells proliferated after hygromycin B selection, therefore, it is quite possible that the ratio increase is the result of proliferation of CD56 positive cells after hygromycin selection.

The mRNA expression of endocrine hormones insulin and glucagons in hygromycin B selected cells from HD437PP and HD438I at P0 are similar to the situations for NeuroD1 and PDX-1. Hygromycin B selected cells not only have higher insulin (30- to 49-fold) and glucagons (27- to 117-fold) to β-actin ratios but also greater absolute mRNA copy numbers than control cells do (Table 1).

TABLE 1
Effect of hygromycin B selection on gene expression of primary
pancreatic cells in culture.

| | HD437PP P0 | | HD438I P0 | |
|---|---|---|---|---|
| | | Hygromycin | | Hygromycin |
| | Control | B | Control | B |
| β-actin mRNA | 792,200 | 212,000 | 597,700 | 142,900 |
| NeuroD1 mRNA | 854 | 22,900 | 21,820 | 52,990 |
| NeuroD1/β-actin | 1.08E−03 | 1.08E−01 | 3.65E−02 | 3.71E−01 |
| Fold Increase | 1 | 110 | 1 | 10 |
| PDX-1 mRNA | 31 | 479 | 1,062 | 6,118 |
| PDX-1/β-actin | 3.85E−05 | 2.26E−03 | 1.78E−03 | 4.28E−02 |
| Fold Increase | 1 | 59 | 1 | 24 |
| Insulin mRNA | 181,800 | 1,463,000 | 4,395,000 | 51,780,000 |
| Insulin/β-actin | 2.30E−01 | 6.90E+00 | 7.35E+00 | 3.62E+02 |
| Fold Increase | 1 | 30 | 1 | 49 |
| Glucagon mRNA | 21,900 | 682,000 | 114,000 | 728,000 |
| Glucagon/β-actin | 2.76E−02 | 3.22E+00 | 1.91E−01 | 5.09E+00 |
| Fold Increase | 1 | 117 | 1 | 27 |
| CD56 mRNA | 7,508 | 13,140 | 17,300 | 2,323 |
| CD56/β-actin | 9.48E−03 | 6.20E−02 | 2.89E−02 | 1.63E−02 |
| Fold Increase | 1 | 6.5 | 1 | 0.56 |
| CK19 mRNA | 97,400 | 127,000 | 119,000 | 7,570 |
| CK19/β-actin | 1.23E−01 | 5.99E−01 | 1.99E−01 | 5.30E−02 |
| Fold Increase | 1 | 4.9 | 1 | 0.27 |

A 6.5-fold increase in CD56 expression from hygromycin B selected HD437PP P0 cells was observed versus non-selected cells. However, a 40% decrease in selected cells from HD438I P0 was observed compared to non-selected control, indicating that hygromycin B selects sub-populations of CD56 expressing cells in the islets where the majority of CD56 expressing cells reside.

The expression of CK19 is similar to that of CD56. CK19 expression in P0 cells from HD437PP was increased about 5-fold while in P0 cells from HD4381 decreased more than 70% after hygromycin B selection.

Expression of Pax4, Nkx2.2, GLUT-2, and glucokinase, was determined in control cells and hygromycin B selected cells from HD437PP P0. Pax4 and Nk×2.2 are transcription factors involved in endocrine cell development especially for insulin expressing β-cells. GLUT-2 is a glucose transpoter on cell surface that transport extracellular glucose into the cytosol of β-cells. Glucokinase is the major glucose phosphorylating enzyme in pancreatic β-cells, and because of its high flux control coefficient on glucose metabolism, it is regarded as the glucose sensor for insulin secretion. Its role in blood glucose homeostasis in humans is evident from mutations in the glucokinase gene, which cause either diabetes (maturity-onset diabetes of the young, type 2) or hypoglycemia. The concerted action of GLUT-2 and glucokinase constitutes the glucose sensing machinery of β-cells. The results are shown in Table 2. The expression levels of Pax4, Nkx2.2, GLUT-2, and glucokinase in HD437PP P0 cells all increased after hygromycin B selection.

TABLE 2
Effects of hygromycin B selection on gene
expression of HD437PP P0 cells.

| | Pax4 | Nkx2.2 | GLUT-2 | Glucokinase |
|---|---|---|---|---|
| Control | 1.53E−05 | 7.74E−06 | 2.51E−05 | 9.65E−05 |
| Hygromycin B | 9.59E−04 | 1.67E−04 | 8.19E−04 | 5.20E−03 |
| Fold Increase | 63 | 21 | 33 | 54 |

P1 cells from HD434 pre-purified layer cultured in medium consisting of 50% SM95 and 50% M3 were used to determine if higher dosages of hygromycin B could be used for selection. Cells were treated with hygromycin B at 50 μg/ml for either 12 or 36 hours. The cells were then maintained in M3+ (see above) for 6 days with one medium change. Control cells were maintained in SM95 at P1 then changed to HJM with supplements at P2. We observed 6 small cell patches which covered about 2% of the surface area of the culture vessel in both conditions. The cells were passaged to 6-well plates and cultured in HJM with supplements for 14 days with three medium changes. The data showed that a one time high dose hygromycin B treatment can achieve similar selectivity as twice low dose treatment in terms of NeuroD1 expression (Table 3).

TABLE 3
Effect of hygromycin B selection on NeuroD1 and insulin expressions
on HD434PP primary pancreatic cells in culture*.

| | Control⁰ | 12 h¹ | 12 h² | 36 h¹ | 36 h² |
|---|---|---|---|---|---|
| NeuroD1/β-actin | 0.0002 | 0.005 | 0.021 | 0.014 | 0.007 |
| Fold Increase | 1 | 25 | 105 | 70 | 35 |
| Insulin/β-actin | 0.0064 | 0.046 | 1.470 | 0.172 | 0.044 |
| Fold Increase | 1 | 7 | 230 | 27 | 7 |

*Data are from P2 cells.
⁰Cells were cultured in HJM supplemented with bFGF (20 ng/ml), betacellulin (10 ng/ml), Nicotinamide (10 mM), and Exendin 4 (1 nM).
¹Cells were cultured in HJM supplemented with bFGF (20 ng/ml), betacellulin (10 ng/ml), Nicotinamide (10 mM), and Exendin 4 (1 nM).
²Cells were cultured in HJM supplemented with bFGF (40 ng/ml), betacellulin (10 ng/ml), Nicotinamide (10 mM), and Exendin 4 (1 nM).

Expression of insulin mRNA was also measured in hygromycin B selected pancreatic endocrine cells from HD437PP and HD4381 at P0. The results were similar to those of NeuroD1. Hygromycin B selected CD56 positive cells had higher insulin to β-actin ratio and greater absolute mRNA copy numbers than control cells (Table 1). Hygromycin B appeared to promote differentiation of CD56 positive endocrine progenitors to insulin expressing cells. Similar results were observed using different cell sources and with low dose Hygromycin B treatment (Table 3).

To determine whether hygromycin B can be used to select both mature and progenitor endocrine cells from pancreatic cells culture beyond P0, hygromycin B at 5 μg/ml was applied to P1 cells from HD470I. Gene expression profiles relevant to endocrine linage were compared from hygromycin B selected and control samples by quantitative RT-PCR assays (Table 4). The expression levels of endocrine hormones (normalized to β-actin expression) in hygromycin B selected pancreatic cells were higher than that of control cells. The expression levels of insulin and somatostatin increased more than 20-fold after selection, whereas the increase in glucagon expression is less than 2-fold. All the transcription factors that are believed to be important for endocrine cell fate decision were up-regulated, ranging from 1.7-fold to 22-fold in hygromycin B selected cells compared to control cells. These results indicate that hygromycin B can be used to select both mature and progenitor endocrine cells from pancreatic cells culture at P1.

TABLE 4
Effect of hygromycin B on expression of endocrine linage genes in HD470I P1 cells.

| | Insulin | Glucagon | Somatostatin | PDX-1 | Pax4 | NeuroD1 | Nkx2.2 | Nkx6.1 | Pax6 |
|---|---|---|---|---|---|---|---|---|---|
| Control | 6.01E−02 | 5.99E−03 | 2.76E−03 | 1.10E−05 | 0 | 2.42E−05 | 1.34E−05 | 1.44E−05 | 1.04E−04 |
| Hygromycin | 1.42E+00 | 1.07E−02 | 8.10E−02 | 9.55E−05 | 3.63E−05 | 5.37E−04 | 1.15E−04 | 2.48E−05 | 1.91E−03 |
| Fold | 23.6 | 1.8 | 29.3 | 8.7 | — | 22.2 | 8.6 | 1.7 | 18.4 |
| Increase | | | | | | | | | |

P0 cells from HD436PP were treated with 50 μg/ml Hygromycin B for 2 days and allowed to recover in medium M3+ (previously described) for six days with one medium change. The cells received a second treatment with Hygromycin B at 10 μg/ml for 1 day and recovered in medium M3+for six days. Five to six patches of cells survived Hygromycin B selection in each 10-cm plate. Cells were harvested and pooled. A majority of the cells were propagated to P1 in SM95 medium containing 20% M3 in 10-cm tissue culture plate. Immunocytochemical analysis of one plate of surviving cells showed that most of these cells were CD56 positive. Cells from a sister plate were collected and analyzed by immunocytochemistry. The cell population that survived Hygromycin B treatment included cells that expressed insulin, glucagons, somatostatin, PDX1, CK19, and nestin. Cells from another plate that received same treatment were analyzed by quantitative RT-PCR and the results are shown in Table 5, P0.

TABLE 5
Expression of Insulin and NeuroD1 in proliferated hygromycin B
selected HD434PP pancreatic cells in culture.

| | | | Fold of |
|---|---|---|---|
| | Insulin/β-actin | NeuroD1/β-actin | Expansion |
| P0 | 1.71 | 0.028 | |
| P1 | 0.26 | 0.004 | 10 |
| A | | | |
| P2 a | 1.60 | 0.054 | 40 |
| P2 b | 0.36 | 0.019 | 40 |
| P2 c | 3.59 | 0.084 | 40 |
| P2 d | 39.71 | 0.067 | 40 |
| P2 e | 52.05 | 0.126 | 40 |
| B | | | |
| P2 | 1.64 | 0.03 | 50 |
| C | | | |
| P2 | 0.67 | 0.018 | 50 |
| P3 M3 | 0.027 | 0.0036 | 200 |
| P3 Co1 I | 0.085 | 0.0037 | 200 |
| P3 SM95/KOSR | 0.17 | 0.0096 | 200 |
| D | | | |
| P2 | 0.0044 | 0.48 | 50 |
| P3 a | 0.007 | 0.007 | 200 |
| P4 a | 0.95 | 0.14 | 800 |
| P3 b | 0.24 | 0.004 | 200 |
| P4 b-1 | 0.08 | 0.014 | 800 |
| P4 b-2 | 0.25 | 0.029 | 800 |
| P4 b-3 | 0.94 | 0.036 | 800 |
| P3 c | 0.299 | 0.008 | 200 |
| P4 c-1 | 0.0033 | 0.0055 | 600 |
| P5 c-1 | 0.0024 | 0.0006 | 2400 |
| P6 c-1 | 0.0015 | 0.0035 | 5760 |
| P7 c-1 | 0.08 | 0.027 | 13824 |
| P4 c-2 | ND | ND | 600 |
| P5 c-2 | 0.0052 | 0.0012 | 600 |
| P6 c-2.1 | 0.014 | 0.0007 | 1440 |
| P7 c-2.1 | 0.094 | 0.034 | 5184 |
| P7 c-2.2 | 0.079 | 0.027 | 5184 |
| P7 c-2.3 | 0.134 | 0.038 | 5184 |
| P6 c-2.2 | 0.248 | 0.012 | 1440 |

### Example 3

### Hygromycin B-Selected, Pancreatic Endocrine Cells can be Expanded

To determine whether hygromycin B selected cells can be expanded, P1 cells from HD436PP were cultured in SM95 containing 20% M3 medium for 10 days with two medium changes. After reaching confluence, cells were harvested and ⅕ of P1 cells were analyzed by quantitative RT-PCR (Table 5). The remainder were split into four equal portions and passed to P2 in four different conditions. In condition A, cells were seeded in a 6-well culture dish. Cells in two of the wells were seeded and cultured in HJM supplemented with 20 ng/ml bFGF for 9 days and switched to either HJM (P2 d) or SM95 (P2 e) with 10 mM nicotinamide supplementation. The others were seeded in SM95 overnight and switched to one of the following media conditions: B (P2 a); HJM/bFGF(20 ng/ml) Betacellulin (10 ng/ml), C(P2 b); SM95 with 10% M3 for one day then SM95 alone, or D (P2 c); SM95/bFGF(20 ng/ml)/FGF10 (10 ng/ml). Cells were maintained in the aforementioned conditions for 15 days with media changes every three days and harvested for quantitative RT-PCR analyses. In condition B, cells were seeded in a new 10-cm plate with SM95 for overnight and switched the medium to HJM supplemented with 20 ng/ml bFGF. In condition C, cells were seeded in a new 10-cm plate and maintained in SM95. In condition D, the cells were seeded into the plate which these cells were cultured at P1 and maintained in SM95 containing 20% M3. Cells in conditions B to D were maintained in the respective medium for about two weeks with four medium changes and harvested for quantitative RT-PCR analysis.

P2 cells cultured in condition A formed aggregates easily, especially when no serum was present in the medium. Quantitative RT-PCR analyses revealed that cells seeded in HJM/bFGF (20 ng/ml) and later maintained in either HJM or SM95 with 10 mM nicotinamide expressed much more insulin mRNA than cells maintained in other conditions (Table 5). It appeared serum has negative impact on these cells in terms of insulin and NeuroD1 mRNA expressions (Table 5, A. P2 b). In most cases, P2 cells in this group expressed more insulin and NeuroD1 than P0 and P1 cells did.

P2 cells cultured in condition B also formed aggregates easily and the aggregates expressed comparable levels of insulin and NeuroD1 mRNAs as that of P0 and P1 cells (Table 5, B. P2). A similar trend was observed for P2 cells cultured in condition C (Table 5, C. P2). A further decrease in the expression of insulin mRNAs was seen when P2 cells were cultured in condition D (SM95 containing 20% M3). Interestingly, the expression levels of NeuroD1 was much higher than not only P0 or P1 cells but also P2 cells cultured in other three conditions (Table 5, D. P2).

P2 cells were expanded and cultured in condition C to passage 3 when cells reached confluence by 1 to 4 split ratio. Equal amounts of cells derived from condition C were seeded in either collagen I or M3 coated cultured dishes and maintained in SM95 medium or regular culture dish and maintained in SM95 supplemented with 1% knockout serum replacement (KOSR) for 2 days and changed to SM95. Cells were cultured for 8 days total, with two medium changes, and harvested. A majority of the cells were banked and some were analyzed by quantitative RT-PCR. No aggregate formation was observed at this stage. In general, mRNA for insulin and NeuroD1 expressed in condition C P3 are less than P2 cells. In addition, cells grown in coated culture dishes had lower levels of insulin and NeuroD1 mRNAs than cells seeded in uncoated dish in SM95 containing 1% KOSR (Table 5, C. P3).

P2 cells cultured in condition D were expanded to passage 3 when cells reached confluence. One quarter of the cells were collected for quantitative RT-PCR analysis. The remainder were equally divided into three portions and cultured in conditions described below. In condition a, cells were plated back into the original dish with SM95 containing 20% M3. In other conditions, cells were seeded in SM95 containing 10% M3 for one day then changed to either HJM/20 ng/ml bFGF (b) or SM95 (c). Cells in condition a reached confluence after 4 days in culture. One-eighth of cells were collected for quantitative RT-PCR analysis and equal amount of cells were passed to P4 in one new plate and cultured in SM95 for 13 days and subsequently either analyzed by quantitative RT-PCR or banked. We cultured condition b cells for 13 days with 3 changes of medium. We collected ¼ of the cells for quantitative RT-PCR analysis and split the remaining cells into three equal portions and maintained in three different conditions as follows: condition b-1 and condition b-2 were seeded in HJM/20 ng/ml bFGF on M3 or collage I coated plate respectively, and condition b-3 in regular culture dish with HJMJ/1%/KOSR initially and switched to HJM/20 ng/ml bFGF the next day. Cells were cultured in these conditions for 8 days with 2 medium changes and harvested for quantitative RT-PCR analysis. Cells cultured in SM95 (condition c) had very limited proliferation after 22 days. The cells were harvested and ⅓ of cells submitted for quantitative RT-PCR analysis. The remainders were passed to passage 4 into two pre-conditioned 10-cm. culture dishes (condition c-1 and c-2) in SM95 containing 20% M3 and maintained in this condition for 8 days with two medium changes.

Quantitative RT-PCR results showed that serum has negative effect on insulin expression in cultured pancreatic cells as previously observed (Table 5, D, a vs b, c). Cells of same passage (P3) or higher passage (P4) cultured in the absence of serum improved their insulin gene expression (Table 5, P3b, P3c, P4a). Coating the surface of culture plates also appeared to influence gene expression in pancreatic cells. In general, cells maintained in either M3 or collagen I coated plates expressed less insulin and NeuroD1 when compared to cells cultured in non-coated plate (Table 5, b-1, b-2, b-3). Cells at P3 and P4 expressed less NeuroD1 than P2 cells (Table 5, D, P2 vs P3, P4).

P4 cells cultured in conditions c-1 and c-2 were harvested when they reached confluence. Half of c-1 cells were frozen down and ¼ of cells were analyzed using quantitative RT-PCR and ¼ of cells were passed to passage 5 in a new culture dish with SM95 (P5 C-1). Cells in condition c-2 were passed to passage 5 in a new culture dish with HJM/20 ng/ml bFGF without splitting (P5 c-1). P5 cells reached confluence in both conditions after 10 days in culture and were harvested. One-sixth of cells from both conditions were submitted for quantitative RT-PCR analysis. The remaining cells maintained in SM95 were split into two portions, one for banking and the other one for propagation to P6 in SM95 in a dish used to grow P5 cells (P6 c-1). The remainder cells which cultured in HJM/20 ng/ml bFGF were propagated to P6 in the same medium under two conditions, one with a new culture dish (P6 c-2.1) and the other one in the dish used for culturing the P5 cells (P6 c-2.2). P6 cells were maintained in respective media for 22 days before harvest for analysis and further propagation to P7 under different conditions. P7 c-1 cells were from P6 c-1 with a split ratio of 1 to 4.5 and cultured in SM95 for 6 days and harvested for quantitative RT-PCR analysis. P6 c-2.1 cells were split at a 1 to 3 ratio and seeded in three pre-conditioned dishes that used to culture P6 c-2.2 (P7 c-2.1), P6 c-1 (P7 c-2.2), and P6 c-2.1 (P7 c-2.3). These cells were cultured in modified Neural Basal medium with Nicotinamide (10 mM), Exendine 4 (5 nM) and non-essential amino acids (IX) supplements for 11 days and harvested for quantitative RT-PCR analysis.

Levels of both insulin and NeuroD1 expression in P4 c-1 cells were lower than cells at P3 stage (Table 5, P4 c-1 vs P3 c). The same pattern was observed in cells from P5 (Table 5, P5 c-1, c-2 vs P3 c). The levels of both insulin and NeuroD1 expression were comparable to cells at P3 stage only when P6 cells were cultured in a pre-conditioned dish with HJM/20 ng/ml bFGF (Table 3, P6 c-2.2 vs P3 c). We also observed that P7 cells in every condition tested expressed more insulin and NeuroD1 than their predecessors (Table 5, P7 c-1 vs P6 c-1; P7 c-2.1, c-2.2, c-2.3 vs P6 C-2.1).

### Example 4

### Hygromycin B-Selected, Pancreatic Endocrine Cells can be Expanded and Transplanted into Mice

Pancreatic cells from B layer or PP layer of HD451 were isolated to select endocrine precursor cells from human pancreatic primary culture at early passage. Cells were seeded in M3 initially and reached confluence after four days of in vitro culture. These cells were split at 1 to 4 ratio to P1 and seeded in SM95/M3 (1:1) for 5 days. At this point, the cells were nearly confluent. These cells were subjected to hygromycin B (5 μg/ml in M3) selection for 72 hours. This selection process killed about 95% of cells. Surviving cells appeared in small patches and were recovered in M3 for 3 days, synchronized in M3 supplemented with LIF (1000 u), ITS (1×), and aphidicolin (4 μg/ml) for 4 days, and subsequently proliferated in M3/SM95 (1:4) for one week. Cells were then split at 1 to 3 ratio from passages 2 to 4 every 5 to 7 days and cultured in SM95. The expansion of cells from P0 to P4 was greater than 100-fold.

After 4 days culture in SM95 at passage 4, cells were cultured in differentiation medium for about 3 weeks and encapsulated in microcapsules (MICs) made from alginate. The MICs were kept in M4 for either 1-2 days or in either M4 or differentiation medium (DM) for two months and then encapsulated in macrocapsules (MACs) made from alginate. The MICMACs were transplanted into diabetic C57B mice induced by STZ. Some of the MICs were characterized by SGS and ICC analysis. Table 6a and b summaries the results of quantitative RT-PCR analysis of hygromycin B selected cells at different passages. Remarkably, the expression of insulin mRNA was maintained at a stable level from P1 to P4 regardless of the origin of cells. Expression of HNF3b, PDX-1, NeuroD1, SA11, glucagon, somatostatin, and CK19 in these cells was also analyzed by quantitative RT-PCR. A modest decrease from P1 to P2 for all markers tested was seen, however, the expression levels were maintained at a relatively stable level from P2 to P4.

TABLE 6a
Effect of hygromycin B on expression of endocrine lineage genes in HD451 cells.

| Cell | Insulin | Glucagon | Somatostatin | PDX1 | NeuroD | Pax4 | Nkx2.2 |
|---|---|---|---|---|---|---|---|
| B P0 | 1.72E+00 | 9.83E−02 | 5.38E−02 | 4.17E−04 | 4.70E−04 | 4.58E−06 | 1.47E−04 |
| PP P0 | 1.90E+00 | 8.74E−02 | 5.28E−02 | 3.09E−04 | 2.56E−04 | 1.14E−06 | 1.21E−04 |
| B P1 | 3.87E−01 | 9.64E−02 | 7.50E−02 | 1.53E−04 | 8.17E−04 | 4.67E−05 | 1.67E−04 |
| PP P1 | 4.41E−01 | 9.30E−02 | 6.21E−02 | 1.40E−04 | 8.11E−04 | 5.56E−05 | 2.05E−04 |
| B P2 | 1.35E−01 | 3.52E−02 | 2.37E−02 | 4.43E−05 | 2.82E−04 | 3.51E−05 | 2.82E−05 |
| PP P2 | 1.08E−01 | 3.16E−02 | 2.39E−02 | 3.10E−05 | 1.65E−04 | 2.03E−05 | 4.43E−05 |
| B P3 | 2.69E−01 | 8.74E−03 | 4.31E−03 | 5.35E−05 | 8.76E−05 | 5.18E−06 | 1.44E−05 |
| PP P3 | 1.57E−01 | 4.57E−03 | 5.12E−03 | 1.47E−05 | 5.36E−05 | 4.54E−06 | 1.02E−05 |
| B P4 | 3.61E−01 | 5.00E−02 | 6.72E−03 | 3.96E−05 | 1.74E−04 | 2.23E−06 | 2.10E−05 |
| PP P4 | 1.35E−01 | 2.67E−03 | 4.38E−03 | 1.38E−05 | 3.26E−05 | 1.15E−06 | 5.26E−06 |
| B P4 DM | 1.72E−01 | 3.12E−03 | 2.66E−03 | 1.71E−05 | 1.04E−05 | 8.88E−07 | 4.43E−06 |
| PP P4 DM | 2.13E−01 | 1.87E−03 | 2.85E−03 | 2.10E−05 | 7.57E−06 | 0.00E+00 | 2.13E−05 |

TABLE 6b

| Cell | Nkx6.1 | Pax6 | HNF3β | GLUT-2 | Glucokinase | CD56 |
|---|---|---|---|---|---|---|
| B P0 | 1.70E−04 | 2.95E−03 | 1.36E−03 | 9.02E−05 | 1.68E−03 | 4.40E−02 |
| PP P0 | 8.38E−05 | 2.27E−03 | 1.05E−03 | 2.22E−04 | 8.66E−04 | 3.28E−02 |
| B P1 | 4.13E−05 | 1.11E−03 | 9.63E−04 | 1.48E−04 | 4.32E−04 | 1.76E−04 |
| PP P1 | 4.03E−05 | 1.18E−03 | 4.07E−04 | 4.23E−04 | 4.25E−04 | 2.91E−04 |
| B P2 | 1.71E−05 | 3.56E−04 | 4.07E−04 | 9.14E−05 | 1.59E−04 | 1.03E−04 |
| PP P2 | 2.31E−05 | 2.89E−04 | 4.25E−04 | 5.64E−05 | 1.03E−04 | 1.91E−04 |
| B P3 | 4.25E−06 | 8.77E−05 | 2.57E−04 | 1.70E−05 | 3.55E−05 | 7.33E−05 |
| PP P3 | 1.62E−05 | 6.96E−05 | 5.05E−04 | 1.97E−06 | 1.21E−05 | 1.37E−04 |
| B P4 | 2.77E−05 | 1.77E−04 | 1.69E−03 | 3.08E−06 | 4.71E−05 | 1.31E−03 |
| PP P4 | 1.21E−07 | 5.80E−05 | 8.93E−05 | 2.09E−08 | 1.14E−05 | 4.16E−04 |
| B P4 DM | 2.16E−06 | 4.97E−05 | 0.00E+00 | 3.32E−06 | 1.39E−05 | 2.50E−04 |
| PP P4 DM | 2.45E−05 | 7.23E−05 | 0.00E+00 | 0.00E+00 | 1.52E−05 | 1.87E−03 |

We repeated this selection scheme using pancreatic cells from three other donors and the results are shown in Table 7. These results are similar to those obtained from HD451B or HD451P experiments (see Table 7).

TABLE 7
Effect of hygromycin B on insulin, PDX-1 and NeuroD
expressions in HD469B, HD470B, and HD471B cells.

| Cell | Insulin | PDX-1 | NeuroD |
|---|---|---|---|
| HD469B P1 | 9.39E+00 | 2.53E−03 | 4.65E−03 |
| HD469B P2 | 6.80E+00 | 3.07E−03 | 3.28E−03 |
| HD469B P3 | 2.15E+00 | 7.19E−04 | 1.08E−03 |
| HD469B P4 | 1.22E+00 | 3.03E−04 | 1.41E−04 |
| HD470B P0 | 2.54E+00 | 3.03E−03 | 1.26E−03 |
| HD470B P1 | 6.59E+00 | 1.11E−03 | 2.54E−03 |
| HD470B P2 | 8.49E−01 | 3.13E−04 | 2.07E−04 |
| HD470B P3 | 4.19E−01 | 1.16E−04 | — |
| HD470B P4 | 4.53E−01 | 4.31E−05 | — |
| HD471B P1 | 3.77E+00 | 2.11E−03 | 3.34E−03 |
| HD471B P2 | 2.15E+00 | 4.31E−03 | 1.08E−03 |
| HD471B P3 | 7.21E−01 | 3.39E−04 | 6.15E−04 |
| HD471B P4 | 2.07E+00 | 1.59E−04 | — |

The hygromycin B selected cells at P4 were further analyzed by immunocytochemistry and results showed that some of them express PDX-1, CK-19, insulin (c-peptide), glucagon, somatostatin, and amylase. Results are summarized in Table 8. Interestingly, cells from B-layer did not express amylase while cells from PP-layer did not express somatostatin. Some encapsulated cells from B layer after two months of in vitro culture in DM expressed PDX-1, insulin (c-peptide), glucagon, amylase, whereas cells from PP layer did not express glucagon. When encapsulated cells (either B- or PP-layer) were cultured in M4 medium some of them expressed PDX-1, insulin (c-peptide), and amylase. One interesting finding was that after encapsulation these cells expressed neither somatostatin nor CKI9.

TABLE 8
Summary of ICC results on HD451 cells.

| HD451 | | | C-Peptide | Glucagon | Somatostatin | Amylase | PDX-1 | CK19 |
|---|---|---|---|---|---|---|---|---|
| Dish (2D) | SM95 | B | + | + | + | − | + | + |
| | | PP | + | +/− | − | + | + | + |
| MIC (3D) | DM | B | + | + | − | + | + | − |
| | | PP | + | − | − | + | + | − |
| | M4 | B | +/− | − | − | + | + | − |
| | | PP | + | − | − | + | + | − |

In an attempt to make improvements on the selection scheme we tried a repetitive selection process. This involves a second selection with higher dose of hygromycin B after either a short or long recovery time following the first selection. We used cells from HD484I as starting material. These cells were selected at P1 using hygromycin B at a concentration of 5 μg/ml and recovered for either 3 days in M3 or 5 days in M3 followed by 5 days in recovery medium as described previously. In the second selection, hygromycin B at 7.5 mg/ml was added to the culture for 4 days and subsequently the cells were cultured in recovery medium till reached confluence. Ensuing treatments of cultures were the same as previously described. We characterized these cells by quantitative RT-PCR assay and the results are shown in Table 9. We found that the expression levels of endocrine hormones and transcription factors important for endocrine cell commitment are all higher at P4 than P0. Expression levels for some of the tested markers such as PDX-1 and Nkx6.1 decreased in P2 and P3 but rebounded to levels higher than P0 at P4.

TABLE 9
Effect of hygromycin B on expression of endocrine linage genes in HD484I cells.

| Cell | Insulin | Glucagon | Somatostain | PDX-1 | NeuroD | Pax4 | Nkx2.2 | Nkx6.1 | CD56 |
|---|---|---|---|---|---|---|---|---|---|
| P0 | 2.54E+00 | 6.94E−03 | 2.14E−02 | 5.16E−04 | 1.62E−04 | 3.61E−06 | 7.13E−05 | 1.85E−04 | 6.37E−03 |
| P1-1* | 5.31E+00 | 1.09E−01 | 5.71E−02 | 4.53E−04 | 1.68E−03 | 1.79E−04 | 3.95E−04 | 1.23E−04 | 5.05E−03 |
| P1-2# | 1.08E+01 | 1.60E−01 | 1.04E−01 | 6.59E−04 | 1.83E−03 | 3.77E−04 | 6.22E−04 | 2.27E−04 | 2.35E−03 |
| P2-1 | 1.94E+00 | 2.04E−02 | 1.75E−02 | 1.11E−04 | 5.71E−04 | 9.92E−05 | 1.12E−04 | 4.00E−05 | 4.39E−03 |
| P2-2 | 2.10E+00 | 2.00E−02 | 1.55E−02 | 8.95E−05 | 5.60E−04 | 1.20E−04 | 8.98E−05 | 2.49E−05 | 6.20E−04 |
| P3-1 | 1.37E−01 | 1.73E−03 | 1.96E−03 | 7.93E−06 | 7.15E−05 | 1.73E−05 | 1.27E−05 | 7.77E−07 | 2.93E−03 |
| P3-2 | 2.25E+00 | 2.76E−02 | 1.71E−02 | 1.12E−04 | 3.90E−04 | 9.86E−05 | 7.07E−05 | 1.75E−05 | 3.26E−04 |
| P4-1 | 2.28E+01 | 4.40E−01 | 4.03E−01 | 2.18E−03 | 9.93E−03 | 5.39E−04 | 2.72E−03 | 1.05E−03 | 7.61E−03 |
| P4-2 | 2.64E+01 | 3.70E−01 | 4.58E−01 | 2.32E−03 | 8.22E−03 | 9.13E−04 | 2.22E−03 | 7.13E−04 | 5.22E−03 |

*Cells were recovered in M3 for 3 days after the first hygromycin B treatment and followed by a second selection.
#Cells were recovered sequentially in M3 and recovery medium for 5 days each after the first hygromycin B treatment and followed by a second selection.

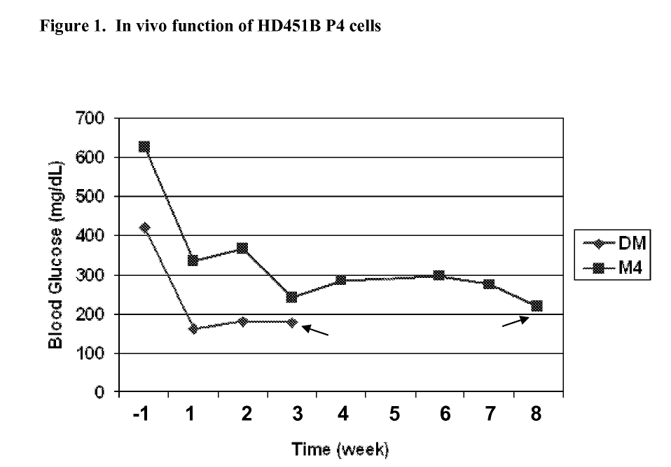

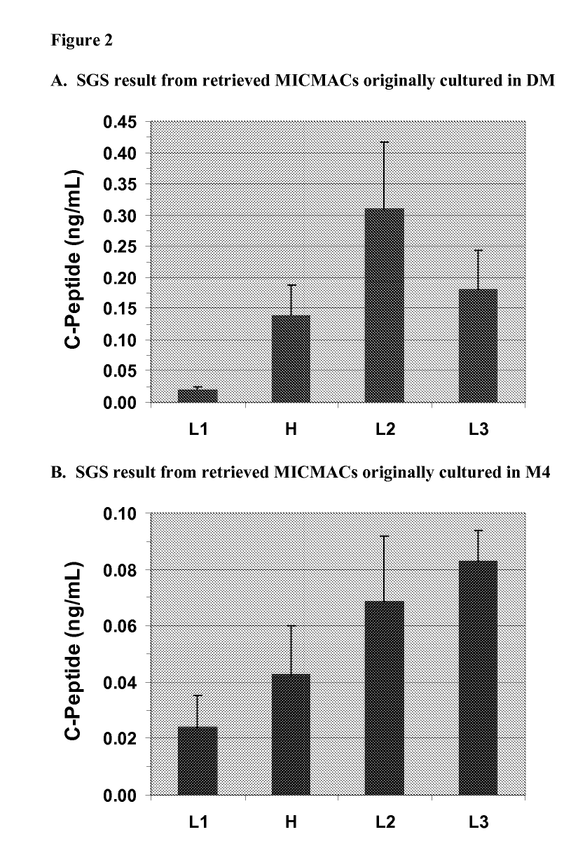

To test the in vitro and in vivo functions of proliferated hygromycin B selected pancreatic cells we did a pilot animal transplantation study using cells from HD451B at P4. Briefly, after 4 days culture in SM95 at P4, cells were cultured in differentiation medium for about 3 weeks and encapsulated in microcapsules (MICs) made from alginate. These MICs were kept in either M4 or differentiation medium (DM) for two months and then encapsulated in macrocapsules (MACs) made from alginate. The MICMACs were transplanted into diabetic C57BL/6 mice induced by single intraperitoneal injection of streptozotocin at 225 μg/Kg body weight. Some of the MICs were characterized by ICC before put into MACs. FIG. 1 shows the results of animal transplantation studies. MICMACs containing HD451B P4 cells were able to lower blood glucose levels in diabetic C57BL/6 mice to either normal or near normal range. These MICMACs were retrieved at either 3-week or 8-week post transplantation (arrows in FIG. 1) and characterized by both ICC and SGS assays. For SGS assay, retrieved MICMACs were placed in 24-well plates and incubated in low glucose medium for 2 hours. These MICMACs were then incubated sequentially in KLB with 60 mg/dL glucose for 1 hour, KLB with 450 mg/dL glucose and 10 mM theophyllin for 1 hour, and two consecutive KLB with 60 mg/dL for 1 hour. The media were collected after each incubations and the amount of C-peptide secreted by the cells inside MICMACs quantified by ELISA. The results are shown in FIG. 2. MICMACs incubated in DM prior to transplantation secret more human C-peptides than those in M4. However, the secretion response to high glucose is delayed. The delayed response may due to the fact that secreted C-peptides diffuse though alginate membranes to reach to the surrounding medium.

### Example 4

### Puromycin can Also be Used to Select for Pancreatic Endocrine Lineage Cells

In an attempt to see if other protein synthesis inhibitors have similar effect as hygromycin B on cultured pancreatic cells puromycin was used in a pilot experiment. We treated P0 cells from HD487B with puromycin at either 2.5 or 5 μg/mL concentrations in SM95/M7 (4:1) for overnight. The cells were recovered in the same medium without puromycin till reaching confluence and passaged to P1. P1 and P2 cells were cultured in SM95/M7 (9:1) with Wnt3a (20 ng/mL). P3 and P4 cells were cultured in SM95/M7 (4:1) with bFGF (50 ng/mL). P5 cells were cultured in maturation medium. We took portions of cells at each passages and analyzed insulin gene expression by quantitative RT-PCR and normalized to β-actin expression. The results are shown in Table 10. Cells that survived puromycin treatment also expressed insulin at passages 1, 2, 3, 4, and 5.

TABLE 10
Effect of puromycin on insulin expression in HD487B cells.

| Cell | Insulin |
|---|---|
| P0 control | — |
| P0 2.5 μg/mL | — |
| P0 5 μg/mL | — |
| P1 control | 3.15E−02 |
| P1 2.5 μg/mL @ P0 | 3.96E−01 |
| P1 5 μg/mL @ P0 | 8.48E−02 |
| P2 control | 6.27E−03 |
| P2 2.5 μg/mL @ P0 | 1.39E−02 |
| P2 5 μg/mL @ P0 | 2.51E−02 |
| P3 control | 1.80E−03 |
| P3 2.5 μg/mL @ P0 | 9.89E−03 |
| P3 5 μg/mL @ P0 | 2.63E−02 |
| P4 control | 8.49E−04 |
| P4 2.5 μg/mL @ P0 | 3.46E−03 |
| P5 control | 1.96E−04 |
| P5 2.5 μg/mL @ P0 | 1.83E−03 |

### Example 5

### G418 can also be Used to Select for Pancreatic Endocrine Lineage Cells

HD522B P0 cells were cultured in M3 for 4 days; G418 sulfate in M3 at the following concentrations (μg/ml) was added to the culture: 0, 5, 25, 50, 100, 200, 500, and 1000. The cells were cultured in the presence of G418 sulfate for 48 h, switched to recovery medium (M3/ITS/LIF) for one week and harvested for quantitative RT-PCR analysis. The control group had to be harvested two days post recovery due to confluence. Cell death was observed in the 500 and 1000 μg/ml groups within 24 hours of treatment. At lower dosages, cell death occurred between 1 and 3 days after switch to recovery medium. Cell proliferation could be observed during the recovery period in most of the conditions except in cultures containing 500 and 1000 μg/ml G418 sulfate. Cell confluence from each plate was estimated as following: 0 (100%), 5 (100%), 25 (95%), 50 (85%), 100 (50%), 200 (20%), 500 (less than 5%), 1000 (less than 2%). Approximately equal amount of cells from each plate were used for analysis based on the estimated confluences of each plate except the plates received 500 and 1000 μg/ml G418 sulfate treatment. Tables 11a-c summarize the ratios of different markers to β-actin from quantitative RT-PCR. The β-actin mRNA copy numbers have no significant variation in control group and cells received 5, 25, 50, and 100 μg/ml G418 whereas cells that received 200 to 1000 μg/ml G418 have much lower β-actin expression especially the 500 and 1000 μg/ml samples. This correlated well with the decreased cell confluence in these samples. The expression of endocrine gene markers increased as the concentration of G418 concentration increased to 100 μg/ml and decreased when G418 concentration is above 200 μg/ml. The expression of amylase, an exocrine marker, and nestin, a marker of mesenchymal cell, decreased as G418 concentration increased.

### Table 11a-c

### Effect of G418 sulfate on gene expression of cultured adult human pancreatic cells.

TABLE 11a

| G418 Sulfate | Insulin/β- | Gcg/β- | SST/β- | Amylase/β- | GLUT2/β- | GCK/β- |
|---|---|---|---|---|---|---|
| (μg/ml) | actin | actin | actin | actin | actin | actin |
| 0 | 1.11E+00 | 1.37E−02 | 2.97E−02 | 8.95E−04 | 1.31E−05 | 1.62E−04 |
| 5 | 1.67E+00 | 1.97E−02 | 5.88E−02 | 1.72E−03 | 4.36E−05 | 2.20E−04 |
| 25 | 3.40E+00 | 4.56E−02 | 1.03E−01 | 6.09E−04 | 1.10E−04 | 2.51E−04 |
| 50 | 5.51E+00 | 9.04E−02 | 1.84E−01 | 4.50E−04 | 2.17E−04 | 3.01E−04 |
| 100 | 1.06E+01 | 1.85E−01 | 4.63E−01 | 5.09E−04 | 4.53E−04 | 8.81E−04 |
| 200 | 1.56E+01 | 5.44E−01 | 1.37E+00 | 1.29E−03 | 1.33E−03 | 2.74E−03 |
| 500 | 8.21E+01 | 3.80E+00 | 8.06E+00 | 7.63E−03 | 1.09E−02 | 1.85E−02 |
| 1000 | 2.02E+02 | 4.98E+00 | 2.43E+01 | 1.24E−02 | 1.29E−02 | 2.39E−02 |

TABLE 11b

| G418 Sulfate | PDX/β- | NeuroD/β- | Nkx2.2/β- | Pax4/β- | Nkx6.1/β- | |
|---|---|---|---|---|---|---|
| (μg/ml) | actin | actin | actin | actin | actin | P48/β-actin |
| 0 | 3.50E−04 | 6.06E−04 | 9.58E−05 | 1.70E−06 | 2.15E−04 | 6.81E−03 |
| 5 | 7.02E−04 | 1.26E−03 | 7.84E−05 | 9.82E−06 | 4.52E−04 | 8.62E−03 |
| 25 | 1.11E−03 | 1.79E−03 | 2.35E−04 | 1.24E−05 | 6.45E−04 | 1.12E−02 |
| 50 | 1.28E−03 | 3.10E−03 | 4.03E−04 | 2.63E−05 | 6.82E−04 | 9.21E−03 |
| 100 | 1.72E−03 | 6.94E−03 | 8.70E−04 | 5.31E−05 | 9.19E−04 | 7.93E−03 |
| 200 | 6.90E−03 | 1.76E−02 | 3.31E−03 | 2.32E−04 | 1.99E−03 | 1.04E−02 |
| 500 | 5.35E−02 | 1.42E−01 | 2.84E−02 | 1.50E−03 | 1.28E−02 | 2.43E−02 |
| 1000 | 4.87E−02 | 1.72E−01 | 6.69E−02 | 7.64E−04 | 1.37E−02 | 5.95E−02 |

TABLE 11C

| G418 Sulfate | NCAM/β- | CK19/β- | Nestin/β- | Delta/β- | Notch1/β- | |
|---|---|---|---|---|---|---|
| (μg/ml) | actin | actin | actin | actin | actin | Ngn3/β-actin |
| 0 | 9.31E−02 | 3.97E−01 | 2.63E−03 | 6.62E−05 | 9.32E−05 | 0.00E+00 |
| 5 | 1.51E−01 | 7.51E−01 | 2.52E−03 | 1.24E−04 | 8.14E−05 | 2.45E−07 |
| 25 | 2.23E−01 | 1.06E+00 | 1.24E−03 | 1.08E−04 | 3.02E−04 | 9.19E−07 |
| 50 | 1.51E−01 | 1.02E+00 | 5.42E−04 | 7.70E−05 | 4.91E−04 | 2.13E−06 |
| 100 | 9.73E−02 | 9.98E−01 | 3.48E−04 | 1.16E−04 | 5.39E−04 | 2.71E−06 |
| 200 | 6.17E−02 | 4.09E−01 | 1.81E−03 | 2.67E−04 | 1.60E−03 | 2.37E−06 |
| 500 | 5.51E−02 | 1.03E−01 | 3.37E−03 | 1.27E−03 | 1.93E−03 | 0.00E+00 |
| 1000 | 3.31E−02 | 1.44E−02 | 0.00E+00 | 4.18E−03 | 3.99E−03 | 0.00E+00 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of isolating a culture of pancreatic endocrine lineage cells, the method comprising:
(a) isolating a culture of pancreatic cells from a pancreas, wherein the culture of isolated pancreatic cells includes a population of pancreatic endocrine lineage cells and a population of pancreatic exocrine cells;
(b) incubating the culture of isolated pancreatic cells with an inhibitor of protein synthesis in an amount that is lethal to at least 30% of the population of pancreatic exocrine cells; and
(c) isolating pancreatic endocrine lineage cells that survive incubation with the inhibitor of protein synthesis to obtain the culture of pancreatic endocrine lineage cells.

2. The method of claim 1, wherein the inhibitor of protein synthesis is an antibiotic.

3. The method of claim 2, wherein the inhibitor of protein synthesis is selected from the group consisting of hygromycin B, puromycin, and G418.

4. The method of claim 1, wherein at least 40% of the isolated pancreatic endocrine lineage cells express CD56 as a cell surface marker.

5. The method of claim 1, wherein at least 80% of the isolated pancreatic endocrine lineage cells express CD56 as a cell surface marker.

6. The method of claim 1, wherein at least 90% of the isolated pancreatic endocrine lineage cells express CD56 as a cell surface marker.

7. The method of claim 1, comprising incubating the culture of isolated pancreatic cells with the inhibitor of protein synthesis in an amount that is lethal to at least 50% of the population of pancreatic exocrine cells.

8. The method of claim 1, comprising incubating the culture of isolated pancreatic cells with the inhibitor of protein synthesis in an amount that is lethal to at least 90% of the population of pancreatic exocrine cells.

9. The method of claim 1, comprising incubating the culture of isolated pancreatic cells with the inhibitor of protein synthesis in an amount that is lethal to at least 95% of the population of pancreatic exocrine cells.

10. The method of claim 1, wherein the culture of pancreatic endocrine cells comprises a stromal cell.

11. The method of claim 1, wherein the culture of isolated pancreatic cells is isolated from a human pancreas.

12. The method of claim 1, further comprising the step of expanding the culture of pancreatic endocrine lineage cells.

13. The method of claim 12, further comprising the step of differentiating the expanded culture of pancreatic endocrine lineage cells.

14. The method of claim 13, further comprising the step of encapsulating the differentiated culture of pancreatic endocrine lineage cells.

15. A method of providing pancreatic endocrine function to a mammal in need of such function, the method comprising the steps of:
(a) isolating a culture of pancreatic cells from a pancreas, wherein the culture of isolated pancreatic cells includes a population of pancreatic endocrine lineage cells and a population of pancreatic exocrine cells;
(b) incubating the culture of isolated pancreatic cells with an inhibitor of protein synthesis in an amount that is lethal to at least 30% of the population of pancreatic exocrine cells; and
(c) isolating pancreatic endocrine lineage cells that survive incubation with the inhibitor of protein synthesis to obtain the culture of pancreatic endocrine lineage cells, and
(d) implanting into the mammal the culture of pancreatic endocrine lineage cells in an amount sufficient to produce a measurable amount of insulin in the mammal.

16. The method of claim 15, further comprising the steps of
(i) expanding the culture of pancreatic endocrine lineage cells;
(ii) differentiating the expanded culture of pancreatic endocrine lineage cells; and
(iii) encapsulating the differentiated culture of pancreatic endocrine lineage cells, before implanting the differentiated culture of pancreatic endocrine lineage cells into a mammal.

17. The method of claim 15, where the mammal is a human.

18. A culture vessel comprising
(a) a tissue culture medium,
(b) a protein synthesis inhibitor, and
(c) a culture of untransfected pancreatic endocrine lineage cells.

19. The culture vessel of claim 18, wherein the inhibitor of protein synthesis is selected from the group consisting of hygromycin B, puromycin, and G418.

20. The culture vessel of claim 18, wherein the pancreatic endocrine cells are human cells.
